(19) United States
(12) Patent Application Publication
(10) Pub. No.: US 2024/0358547 A1
(43) Pub. Date: Oct. 31, 2024

(54) MEDICAL PENETRATION AND DRAINAGE FOR GLAUCOMA TREATMENT

(71) Applicant: Beijing Sightnovo Medical Technology Co., Ltd., Beijing (CN)

(72) Inventors: Chan ZHAO, Beijing (CN); Chaoran XIA, Beijing (CN); Chuan LI, Beijing (CN); Yueguang SUN, Beijing (CN)

(21) Appl. No.: 18/285,477

(22) PCT Filed: May 12, 2022
(86) PCT No.: PCT/CN2022/092563

§ 371 Date: Oct. 3, 2023

(30) Foreign Application Priority Data
May 13, 2021 (WO) PCT/CN2021/093650

(51) Int. Cl. A61F 9/007

(52) U.S. Cl. CPC A61F 9/00781; A61F 2250/0067

(57) ABSTRACT

A method and system for treating glaucoma can include positioning an intraocular shunt in eye tissue such that the shunt conducts fluid from the anterior chamber to a target outflow region in the eye, such as the suprachoroidal space and/or the subconjunctival space (or outside the conjunctiva).

### CROSS-REFERENCE TO RELATED APPLICATION

This application claims priority to International Patent Application No. PCT/CN2021/093650, filed May 13, 2021, entitled “Medical Penetration and Drainage for Glaucoma Treatment,” which is herein incorporated by reference in its entirety for all purposes.

### FIELD

The present disclosure in some aspects relates to the field of medical device and apparatus, and specifically a device, kit, assembly, or system for medical penetration and drainage.

### BACKGROUND

Glaucoma is a disease of the eye that affects millions of people. Glaucoma is associated with an increase in intraocular pressure resulting either from a failure of a drainage system of an eye to adequately remove aqueous humor from an anterior chamber of the eye or overproduction of aqueous humor by a ciliary body in the eye. Build-up of aqueous humor and resulting intraocular pressure may result in irreversible damage to the optic nerve and the retina, which may lead to blindness. Generally, glaucoma may be treated by surgical intervention. However, improved methods are still needed. The present disclosure addresses these and other needs.

### SUMMARY

To address at least one of the defects or shortcomings in existing devices and methods, the present disclosure in some aspects provides a kind of medical puncturing device and a medical kit, assembly, or system for medical penetration, which can achieve injection, access, expansion, and/or device implantation (such as implanting a shunt) in the suprachoroidal space or the subconjunctival space as a target outflow region, or between target outflow region and an inflow region (e.g., the anterior chamber). The present disclosure is especially useful for achieving precise control of puncturing depth and needle placement, steady injection and injection of a defined volume, as well as providing improved methods for placing shunts that facilitate drainage of fluid from the anterior chamber.

In some embodiments, provided herein is a method for placing a shunt into an eye, comprising: (a) inserting a needle into the eye to form a delivery passageway in the eye, wherein the delivery passageway ends in a target outflow region in the eye; (b) delivering a flowable composition through the needle to form an expanded space in the target outflow region; (c) positioning an inflow end of a shunt in the anterior chamber of the eye and an outflow end of the shunt in the expanded space, wherein the shunt is releasably coupled to the needle; and (d) releasing the needle from the shunt, thereby placing the shunt in the eye to provide fluid communication between the anterior chamber and the target outflow region.

In some embodiments, the needle pierces the sclera. In any of the embodiments herein, the method can comprise cutting open a region in the conjunctiva, optionally prior to the needle piercing the sclera. In any of the embodiments herein, the needle can pierce the conjunctiva and the sclera, and wherein the method does not comprise cutting open a region in the conjunctiva. In any of the embodiments herein, the target outflow region can be between the sclera and the choroid/ciliary body, and the expanded space can be a suprachoroidal space. In any of the embodiments herein, the positioning step can comprise positioning a distal end of the needle in the suprachoroidal space and towards the anterior chamber angle.

In any of the embodiments herein, the shunt can be within the needle, optionally wherein the shunt is in a needle body passageway of the needle, or the shunt can form a sleeve around the needle.

In any of the embodiments herein, the positioning step can comprise advancing the shunt in/around the needle to a distal end of the needle. In any of the embodiments herein, the advancing can comprise pushing the shunt in/around the needle using a guidewire. In any of the embodiments herein, the positioning step can comprise piercing the anterior chamber angle with a distal end of the needle and/or the shunt. In any of the embodiments herein, the releasing step can comprise removing the needle and/or the guidewire from the eye, leaving the inflow end of the shunt in the anterior chamber and the outflow end of the shunt in the suprachoroidal space.

In any of the embodiments herein, the shunt can be coupled to the needle prior to or after the inserting step. In any of the embodiments herein, the shunt can be coupled to the needle prior to or after delivering the flowable composition. In any of the embodiments herein, the shunt can be releasably coupled to a distal end of the needle.

In any of the embodiments herein, the positioning step can comprise positioning the shunt towards the anterior chamber angle. In any of the embodiments herein, the positioning step can comprise advancing the needle to pierce the anterior chamber angle with a distal end of the shunt. In any of the embodiments herein, the releasing step can comprise removing the needle, leaving the inflow end of the shunt in the anterior chamber and the outflow end of the shunt in the suprachoroidal space.

In any of the embodiments herein, the positioning step can comprise positioning a distal end of the needle in the suprachoroidal space and away from the anterior chamber angle. In any of the embodiments herein, the shunt can be within the needle, optionally wherein the shunt is in a needle body passageway of the needle, or the shunt can form a sleeve around the needle. In any of the embodiments herein, the positioning step can comprise advancing the shunt in/around the needle to the distal end of the needle. In any of the embodiments herein, the advancing can comprise pushing the shunt in/around the needle using a guidewire. In any of the embodiments herein, the positioning step can comprise positioning the outflow end of the shunt in the suprachoroidal space and away from the anterior chamber angle. In any of the embodiments herein, the positioning step can comprise removing the needle from the eye, leaving the outflow end of the shunt in the suprachoroidal space.

In any of the embodiments herein, the method can further comprise piercing the anterior chamber angle to form an implant passageway. In any of the embodiments herein, the inflow end of the shunt can be positioned through the implant passageway in the anterior chamber. In any of the embodiments herein, the implant passageway can be formed using the same needle or a different piercing element. In any of the embodiments herein, the same needle or different piercing element can pierce through the conjunctiva, the sclera, the suprachoroidal space, and the anterior chamber angle. In any of the embodiments herein, the needle can be inserted into the eye at a first entry point, and the same needle or different piercing element can be inserted into the eye at a second entry point different from the first entry point to form the implant passageway.

In any of the embodiments herein, the shunt can comprise a portion between the first and second entry points that is outside the sclera. In any of the embodiments herein, the shunt can comprise a portion between the first and second entry points that is outside the sclera and the conjunctiva. In any of the embodiments herein, the portion outside the sclera can be subconjunctival. In any of the embodiments herein, the method can comprise cutting an opening in the conjunctiva, dissection of the conjunctiva from the sclera to form a conjunctiva flap, and after the inflow end of the shunt is positioned through the implant passageway in the anterior chamber, sewing the opening of the conjunctival flap to cover the portion of the shunt outside the sclera. In any of the embodiments herein, the portion of the shunt outside the sclera can comprise a shunt body outflow port, optionally wherein the shunt body outflow port is subconjunctival.

In any of the embodiments herein, the method can comprise applying an antimetabolite between the conjunctival flap and the sclera to modulate postoperative scarring.

In any of the embodiments herein, the needle can be inserted into the eye ab externo or ab interno.

In any of the embodiments herein, the target outflow region can be between the conjunctiva and the sclera, and the expanded space can be a subconjunctival space, optionally wherein the subconjunctival space is a bleb. In any of the embodiments herein, the shunt can be within the needle, optionally wherein the shunt is in a needle body passageway of the needle; the shunt can form a sleeve around the needle; or the shunt can be releasably coupled to a distal end of the needle.

In some aspects, disclosed herein is a method for placing a shunt into an eye, comprising: (a) inserting a needle into the eye to form a delivery passageway in the eye, wherein the delivery passageway ends between the sclera and the choroid/ciliary body of the eye; (b) delivering a flowable composition through the needle to form a suprachoroidal space; (c) piercing the anterior chamber angle of the eye with a distal end of the needle and/or a shunt releasably coupled thereto; (d) positioning the shunt through the anterior chamber angle such that an inflow end of the shunt is in the anterior chamber and an outflow end of the shunt is in the suprachoroidal space; and (e) removing the needle from the eye, thereby placing the shunt in the eye to provide fluid communication between the anterior chamber and the suprachoroidal space.

In some aspects, disclosed herein is a method for placing a shunt into an eye, comprising: (a) inserting a needle into the eye to form a delivery passageway in the eye, wherein the delivery passageway ends between the sclera and the choroid/ciliary body of the eye; (b) delivering a flowable composition through the needle to form a suprachoroidal space; (c) using the needle to position an outflow end of a shunt in the suprachoroidal space and away from the anterior chamber angle; (d) piercing the anterior chamber angle of the eye to form an implant passageway; and (e) positioning an inflow end of the shunt in the anterior chamber through the implant passageway, thereby placing the shunt in the eye to provide fluid communication between the anterior chamber and the suprachoroidal space.

In any of the embodiments herein, a portion of the shunt can be outside the sclera. In any of the embodiments herein, the portion of the shunt outside the sclera can be subconjunctival. In any of the embodiments herein, a portion of the shunt can be outside the sclera and the conjunctiva. In any of the embodiments herein, the shunt can comprise a shunt body outflow port that is outside the sclera and subconjunctival and/or a shunt body outflow port that is outside the conjunctiva. In any of the embodiments herein, the shunt can provide fluid communication between the anterior chamber and the suprachoroidal space, and between the anterior chamber and a subconjunctival space. In any of the embodiments herein, the shunt can provide fluid communication between the anterior chamber and the suprachoroidal space, and between the anterior chamber and a space outside the conjunctiva.

In some aspects, disclosed herein is an ab interno method for placing a shunt into an eye, comprising: (a) inserting a needle and/or a shunt releasably coupled thereto through the cornea, across the anterior chamber, and to a suprachoroidal space or a subconjunctival space; (b) delivering a flowable composition through the needle and/or the shunt into the suprachoroidal space or the subconjunctival space; (c) positioning an inflow end of the shunt in the anterior chamber and an outflow end of the shunt in the suprachoroidal space or the subconjunctival space; and (d) removing the needle from the eye, thereby placing the shunt in the eye to provide fluid communication between the anterior chamber and the suprachoroidal space or the subconjunctival space.

In any of the embodiments herein, the shunt can comprise a pharmaceutical or biological agent.

In any of the embodiments herein, the method can comprise using a device comprising: a syringe barrel comprising a proximal end and a distal end; a floating seal in the syringe barrel; a needle base proximal to the floating seal, wherein the floating seal and the needle base elastically engage each other; and the needle. In any of the embodiments herein, the needle can comprise: (i) a needle proximal end engaging the needle base; (ii) a needle distal end; (iii) a needle distal opening; (iv) a needle body opening between the needle proximal end and the needle distal end, wherein the needle body opening is proximal to the needle distal opening; and (v) a needle body passageway connecting the needle distal opening and the needle body opening. In any of the embodiments herein, the needle base can be configured to advance the needle distally toward and/or through the floating seal. In any of the embodiments herein, the floating seal can separate a proximal lumen and a distal lumen in the syringe barrel, and wherein the distal lumen comprises the flowable composition. In any of the embodiments herein, the needle base can be configured to advance the needle distally such that the needle distal opening is in the sclera, whereas the needle body opening is in the distal lumen comprising the flowable composition.

In any of the embodiments herein, the sclera may prevent discharge of the flowable composition in the distal lumen through the needle distal opening, optionally wherein the back pressure at the needle distal opening in the sclera is no less than the pressure in the distal lumen. In any of the embodiments herein, the needle base can be configured to advance the needle distally such that the needle body opening is in the distal lumen while the needle distal opening is between the sclera and an adjacent tissue. In any of the embodiments herein, the needle distal opening can be between the sclera and the choroid/ciliary body, and the flowable composition can be delivered through the needle to the suprachoroidal space. In any of the embodiments herein, the needle distal opening can be between the sclera and the conjunctiva, and the flowable composition can be delivered through the needle to the subconjunctival space.

In any of the embodiments herein, the flowable composition can comprise a liquid, a solution, a suspension, a gel, an oil, an ointment, an emulsion, a cream, a foam, a lotion, and/or a paste. In any of the embodiments herein, the shunt can be configured to advance distally through or along the needle and be exposed at a distal end of the needle when the needle reaches the target outflow region.

In some aspects, disclosed herein is a system comprising the needle, the shunt, and the flowable composition for use in the method of any of the embodiments herein.

In some aspects, disclosed herein is a system for placing a shunt into an eye, comprising: a syringe barrel comprising a proximal end and a distal end; a floating seal in the syringe barrel; a needle base proximal to the floating seal, wherein the floating seal and the needle base elastically engage each other; a needle for insertion into the eye, the needle comprising: (i) a needle proximal end engaging the needle base; (ii) a needle distal end; (iii) a needle distal opening; (iv) a needle body opening between the needle proximal end and the needle distal end, wherein the needle body opening is proximal to the needle distal opening; and (v) a needle body passageway connecting the needle distal opening and the needle body opening, wherein the needle base is configured to advance the needle distally toward and/or through the floating seal; and the shunt configured to releasably couple to the needle. In some embodiments, the shunt is an intraocular shunt within the needle.

In any of the embodiments herein, the shunt can comprise a solid structure, a porous structure, a multi-layer composite structure, a membrane stent structure, or any combination thereof. In any of the embodiments herein, the shunt can comprise one or more annular rings on a sidewall of the shunt. In any of the embodiments herein, the shunt can comprise a marker ring and/or a retention ring on the sidewall of the shunt. In any of the embodiments herein, the shunt can comprise multiple channels each extending from one end of the shunt to the other end.

### BRIEF DESCRIPTION OF THE DRAWINGS

The drawings illustrate certain embodiments of the features and advantages of this disclosure. These embodiments are not intended to limit the scope of the appended claims in any manner.

FIGS. 1A-1E show schematic diagrams of the different stages of operating an exemplary medical puncturing device, for example, during the punctuation and injection into a suprachoroidal space (SCS) 14. FIG. 1F show steps of operating an exemplary medical puncturing device without a contacting member (e.g., 1b shown in FIGS. 1A-1E), where a distal seal (e.g., 8 shown in FIGS. 1A-1E) may directly contact a tissue.

FIGS. 2A-2E show schematic diagrams of the different stages of operating an exemplary medical puncturing device, for example, during the punctuation and injection into a suprachoroidal space (SCS) 14. FIG. 2F show steps of operating an exemplary medical puncturing device without a contacting member (e.g., 1b shown in FIGS. 2A-2E), where a distal seal (e.g., 8 shown in FIGS. 2A-2E) may directly contact a tissue.

FIGS. 3A-3F are partial structure diagrams of exemplary medical puncturing devices comprising floating seal 3 and one or more needle body openings (6b or 6b1, 6b2, and/or 6b3) and needle distal opening 6a.

FIGS. 4A-4C are partial structure diagrams of exemplary medical puncturing devices comprising floating seal 3 and needle body opening 6b.

FIGS. 5A-5F are partial structure diagrams of exemplary medical puncturing devices comprising floating seals 3a and 3b and one or more needle body openings (6b or 6b1 and/or 6b2).

FIG. 6 shows a partial structure diagram of an exemplary medical puncturing device comprising a through angled guiding groove 3a and one-way valve 9.

FIG. 7 shows a partial structure diagram of an exemplary medical puncturing device comprising a through angled guiding groove 3a and one-way valve 9.

FIG. 8 shows a partial structure diagram of an exemplary medical puncturing device comprising a non-through angled guiding groove 3a.

FIG. 9 shows a partial structure diagram of an exemplary medical puncturing device comprising an angled guiding needle hole 6c and one-way valve 9.

FIG. 10 shows a partial structure diagram of an exemplary medical puncturing device comprising an angled guiding needle hole 6c and needle hole plug 10.

FIGS. 11A-11B show schematic diagrams of implanting catheter 11 into SCS 14 using an exemplary medical apparatus assembly comprising a central guiding groove 2c. FIG. 11A shows contacting member 1b that contacts a tissue, while FIG. 11B shows distal seal 8 that contacts a tissue without an intervening contacting member.

FIG. 12 shows an exemplary ab externo method for placing a shunt in an eye.

FIGS. 13A-13B show exemplary ab externo methods for placing a shunt in an eye.

FIGS. 14A-14B show exemplary ab interno methods for placing a shunt in an eye.

FIGS. 15A-15C show schematic diagrams of the different stages of operating an exemplary medical puncturing device.

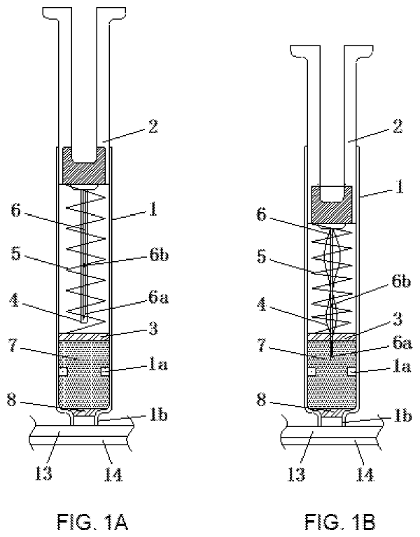

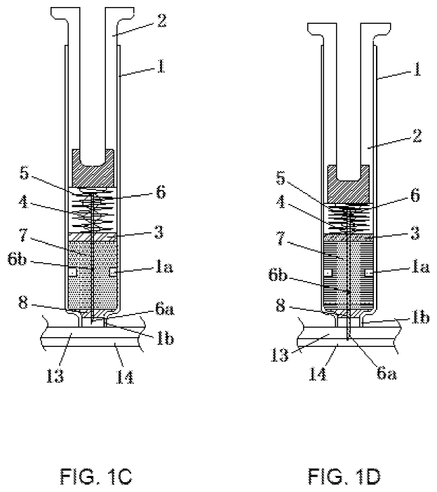

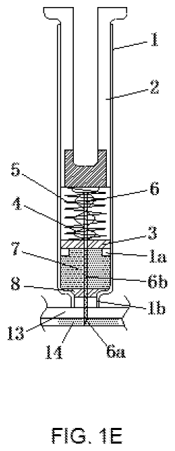

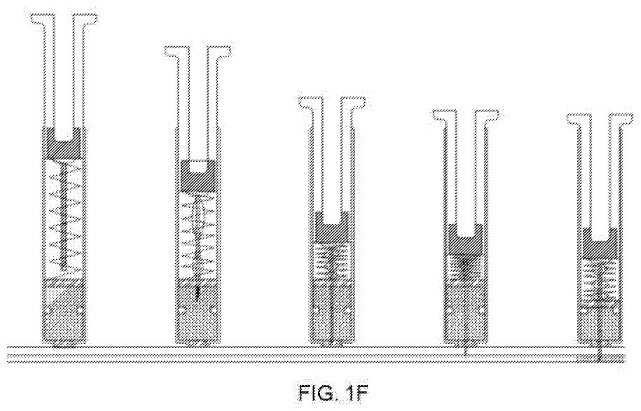

Reference numerals and exemplary corresponding structures are provided below for illustration only, for instance, with reference to FIGS. 1A-1E through FIGS. 11A-11B, and should not be considered limiting: 1—syringe barrel; 1a—axial stopper; 1b—circular contacting element; 2—pressing element; 2c—central guiding groove; 3—floating seal; 3a—angled guiding groove; 4—elastic sheath; 5—spring; 6—hollow puncture needle; 6a—needle distal opening; 6b—needle body opening; 6c—angled guiding needle hole; 7—flowable composition lumen; 8—distal seal; 9—one-way valve; 10—needle hole plug; 11—catheter; 12—auxiliary guiding needle; 13—sclera; 14—suprachoroidal space (SCS).

FIG. 16 shows schematic diagrams of various elements and features of an exemplary medical puncturing device. For instance, the device can comprise a hollow housing 22 engaging a proximal control knob 17. A pressing/push shaft 2 slidably passes through the control knob and engages a guide tube 16 inside the housing. The pressing/push shaft 2 is configured to provide a distally directed force on a compression spring 5, which in turn serves as a force element configured to provide a distally directed force on a piston rod 15. A beveled needle 6, is attached and fixed to a needle base or seat fixed to the pressing/push shaft. The distal end of the needle 6 can reside within a lumen of the piston rod 15 and move distally when a force is applied to move the pressing/push shaft distally. The distal end of the needle can be advanced to pass through a seal 3 at the distal end of the piston rod 15 into a lumen formed by a syringe barrel of a syringe 1 and a distal seal 8. A gland 23 may engage both the syringe 1 and the distal seal 8 to facilitate a sealing engagement. The distal seal 8 can interface a tissue, and the needle 6 can be advanced to pass through the distal seal 8 to penetrate the tissue. The needle 6 may comprise a needle distal opening and a needle body opening, similar to 6a and 6b respectively, as shown and used as described in FIGS. 1A-1E through FIGS. 11A-11B.

FIGS. 17A-17F show schematic diagrams of the different stages of operating an exemplary medical puncturing device.

Reference numerals and exemplary corresponding structures are provided below for illustration only, for instance, with reference to FIG. 17 through FIGS. 17A-17F, and should not be considered limiting: 1—syringe having a syringe barrel forming a lumen; 2—pressing element (e.g., pressing shaft); 3—floating seal (e.g., plunger seal); 5—elastic element such as a spring; 6—hollow puncture needle (needle distal opening and needle body opening not shown); 8—distal seal; 15—piston rod (e.g., push rod); 16—guide tube; 17—control knob; 18—limiter; 19—ruler; 20—adapter; 21—handle; 22—housing; 23—gland.

FIGS. 18A-18C show schematic diagrams of exemplary shunt structures.

FIGS. 19A-19D show additional schematic diagrams of exemplary shunt structures, including a uniform shunt, a shunt comprising a porous material, a shunt comprising multiple layers, and a shunt comprising support covered by a membrane.

FIGS. 20A-20B show schematic diagrams of features of exemplary containers that can be prefilled with a flowable composition and installed in the device.

### DETAILED DESCRIPTION

Below is a detailed description of some embodiments of the present disclosure. It should be understood that the specific implementations described herein are meant to illustrate and explain the embodiments of the present disclosure, and should not be considered limiting.

It should be noted that, when not in conflict, the embodiments of the present disclosure and the features of the embodiments may be combined in any suitable manner.

In some embodiments, the positional descriptions of “front,” “back,” “forward,” “backward,” “distal,” and “proximal,” etc. are based on the perspective of an operator of the medical puncturing device or medical apparatus assembly. That is, when the operator is using the medical puncturing device or medical apparatus assembly, the direction pointing away and relatively far from the operator is the forward direction, and the direction pointing toward and relatively close to the operator is the backward direction.

As used herein, the words “proximal” and “distal” refer to the direction closer to and away from, respectively, an operator (e.g., surgeon, physician, nurse, technician, etc.) who would insert the medical device into the patient, with the tip-end (distal end) of the device inserted inside a patient's body first. Thus, for example, the end of a needle (e.g., microneedle) described herein first inserted inside the patient's body would be the distal end, while the opposite end of the needle (e.g., the end of the medical device being manipulated by the operator) would be the proximal end of the needle.

As used herein, the singular forms “a,” “an,” and “the” include plural referents unless the context clearly dictates otherwise. For example, “a” or “an” means “at least one” or “one or more.” Likewise, the term “a member” is intended to mean a single member or a combination of members, “a material” is intended to mean one or more materials, or a combination thereof.

The term “about” or “approximately” as used herein refers to the usual error range for the respective value readily known to the skilled person in this technical field. Reference to “about” a value or parameter herein includes (and describes) embodiments that are directed to that value or parameter per se. For example, “about” can mean within 1 or more than 1 standard deviation, per the practice in the relevant field. Alternatively, “about” can mean a range of up to 20%, up to 10%, up to 5%, or up to 1% of a given value.

Throughout the present disclosure, various aspects are presented in a range format. It should be understood that the description in range format is merely for convenience and brevity and should not be construed as an inflexible limitation on the scope of the present disclosure. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the present disclosure. The upper and lower limits of these smaller ranges may independently be comprised in the smaller ranges, and are also encompassed within the present disclosure, subject to any specifically excluded limit in the stated range. Where the stated range comprises one or both of the limits, ranges excluding either or both of those comprised limits are also comprised in the present disclosure. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

As used herein, the terms “puncture member”, and “puncturing member” are used interchangeably to refer to an article configured to pierce tissue layers and deliver a substance to a target tissue layer, for example, a needle or a microneedle.

As used herein, the terms “medicament container”, and “medicament containment chamber” are used interchangeably to refer to an article (e.g., a syringe) configured to contain a volume of a substance, for example, a medicament or drug.

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

There are two main types of glaucoma, “open angle” and “closed angle” glaucoma. Open angle glaucoma refers to glaucoma cases in which intraocular pressure increases but an anterior chamber angle (drainage angle) of an eye remains open. A common cause of open angle glaucoma is blockage in the trabecular meshwork, the fluid flow pathways that normally drain aqueous humor from the anterior chamber of the eye. Closed angle glaucoma refers to glaucoma cases in which intraocular pressure increases due to partial or complete closure of the anterior chamber angle. In closed angle glaucoma, swelling or movement of the iris closes the anterior chamber angle and blocks fluid from accessing to the trabecular meshwork, which in turn obstructs outflow of the aqueous humor from the eye.

Glaucoma may be treated by surgical intervention that involves placing a shunt in the eye to result in production of fluid flow pathways between the anterior chamber and various structures of the eye involved in aqueous humor drainage (e.g., Schlemm's canal, the sclera, or the subconjunctival space). Such fluid flow pathways allow for aqueous humor to exit the anterior chamber. The surgical intervention to implant the shunt can involve inserting into the eye a delivery device that holds an intraocular shunt, and deploying the shunt within the eye.

In some embodiments, a delivery device holding the shunt enters the eye through a cornea (ab interno approach), and is advanced across the anterior chamber. The delivery device is advanced through the sclera until a distal portion of the device is in proximity to a drainage structure of the eye. The shunt is then deployed from the delivery device, producing a conduit between the anterior chamber and various structures of the eye involved in aqueous humor drainage (e.g., Schlemm's canal, the sclera, or the subconjunctival space). In some embodiments, a delivery device holding the shunt enters the eye using an ab externo approach, which involves insertion through the conjunctiva of the eye.

Provided herein in one aspect is a method for placing a shunt into an eye, comprising: (a) inserting a needle into the eye to form a delivery passageway in the eye, wherein the delivery passageway ends in a target outflow region in the eye; (b) delivering a flowable composition through the needle to form an expanded space in the target outflow region; (c) positioning an inflow end of a shunt in the anterior chamber of the eye and an outflow end of the shunt in the expanded space, wherein the shunt is releasably coupled to the needle; and (d) releasing the needle from the shunt, thereby placing the shunt in the eye to provide fluid communication between the anterior chamber and the target outflow region. The needle can be inserted into the eye ab externo or ab interno.

In one aspect, provided herein an ab externo method for placing a shunt into an eye, comprising: (a) inserting a needle into the eye to form a delivery passageway in the eye, wherein the delivery passageway ends between the sclera and the choroid/ciliary body of the eye; (b) delivering a flowable composition through the needle to form a suprachoroidal space; (c) piercing the anterior chamber angle of the eye with a distal end of the needle and/or a shunt releasably coupled thereto; (d) positioning the shunt through the anterior chamber angle such that an inflow end of the shunt is in the anterior chamber and an outflow end of the shunt is in the suprachoroidal space; and (e) removing the needle from the eye, thereby placing the shunt in the eye to provide fluid communication between the anterior chamber and the suprachoroidal space.

In another aspect, disclosed herein is an ab externo method for placing a shunt into an eye, comprising: (a) inserting a needle into the eye to form a delivery passageway in the eye, wherein the delivery passageway ends between the sclera and the choroid/ciliary body of the eye; (b) delivering a flowable composition through the needle to form a suprachoroidal space; (c) using the needle to position an outflow end of a shunt in the suprachoroidal space and away from the anterior chamber angle; (d) piercing the anterior chamber angle of the eye to form an implant passageway; and (e) positioning an inflow end of the shunt in the anterior chamber through the implant passageway, thereby placing the shunt in the eye to provide fluid communication between the anterior chamber and the suprachoroidal space.

In yet another aspect, disclosed herein is an ab interno method for placing a shunt into an eye, comprising: (a) inserting a needle and/or a shunt releasably coupled thereto through the cornea, across the anterior chamber, and to a suprachoroidal space; (b) delivering a flowable composition through the needle and/or the shunt into the suprachoroidal space; (c) positioning an inflow end of the shunt in the anterior chamber and an outflow end of the shunt in the suprachoroidal space; and (d) removing the needle from the eye, thereby placing the shunt in the eye to provide fluid communication between the anterior chamber and the suprachoroidal space. In another aspect, disclosed herein is an ab interno method for placing a shunt into an eye, comprising: (a) inserting a needle and/or a shunt releasably coupled thereto through the cornea, across the anterior chamber, and to a subconjunctival space; (b) delivering a flowable composition through the needle and/or the shunt into the subconjunctival space; (c) positioning an inflow end of the shunt in the anterior chamber and an outflow end of the shunt in the subconjunctival space; and (d) removing the needle from the eye, thereby placing the shunt in the eye to provide fluid communication between the anterior chamber and the subconjunctival space.

In some aspects, the methods and devices disclosed herein are useful for minimally invasive glaucoma surgical treatment.

Other features and advantages of the present disclosure will be described in the detailed description below.

Some embodiments of the present disclosure will be described with reference to the several views of the accompanying drawings.

### II. Systems and Devices

In some embodiments, described herein are systems and devices to assist in the insertion of a puncture member, for example, a needle or microneedle into the eye, and/or assist in injecting a medicament into a target ocular tissue. In some embodiments, described herein are systems and devices for controlling the insertion depth of a puncture member, such as, for example, a microneedle, into the eye to deliver a therapeutic agent to, for example, a posterior region of the eye (e.g., via the suprachoroidal space). In some embodiments, described herein are systems and devices for introducing an implant into a tissue, such as an apparent or potential tissue void, cavity, or vessel.

In some embodiments, provided herein is a system comprising a syringe barrel comprising a proximal end and a distal end; a floating seal in the syringe barrel; a needle base proximal to the floating seal (e.g., the needle base is closer to an operator while the floating seal is closer to a subject), and the floating seal and the needle base are configured to elastically engage each other. In some embodiments, the system further comprises a needle comprising a needle proximal end and a needle distal end, and the needle proximal end engages the needle base. In any of the embodiments herein, the needle proximal end can be fixed to the needle base or releasably attached to (e.g., inserted in) the needle base. In any of the embodiments herein, the needle can comprise: (i) a needle distal opening, (ii) a needle body opening between the needle proximal end and the needle distal end, and (iii) a needle body passageway connecting the needle distal opening and the needle body opening. In any of the embodiments herein, the needle body opening can be proximal to the needle distal opening. In any of the embodiments herein, the needle base can be configured to advance the needle distally toward the floating seal (e.g., when the needle distal end is proximal to the floating seal), through the floating seal (e.g., when the needle distal end has entered or pierced into the floating seal), and/or through the distal end of the syringe barrel.

In any of the embodiments herein, a proximal lumen and a distal lumen can be provided in the syringe barrel on different sides of the floating seal. In some embodiments, the distal lumen comprises a flowable composition (e.g., a medicament, a drug, and/or a pharmaceutically acceptable carrier or excipient such as a saline), while the proximal lumen does not contain a non-gas flowable composition. The proximal lumen may be pre-filled with a gas, such as a sterilized air, and/or capable of communicating with the outside environment such as the atmosphere when the needle is advanced in and/or through the syringe barrel.

In some embodiments, the needles included in the embodiments described herein comprise a bevel, which allows for ease of penetration into a tissue such as the sclera and/or suprachoroidal space with minimal collateral damage. In some embodiments, the needles disclosed herein can define a narrow lumen (e.g., gauge size greater than or equal to 30 gauge, 32 gauge, 34 gauge, 36 gauge, etc.) to allow for suprachoroidal drug delivery while minimizing the diameter of the needle track caused by the insertion of the needle. In some embodiments, the lumen and bevel aspect ratio of the needles described herein are the same or different from standard 27 gauge and 30 gauge needles commonly used for intraocular injection.

In some embodiments, a device disclosed herein comprises or is configured to be coupled to a medicament container containing a medicament, such as a gel or the like. The medicament container can be formed at least in part by the syringe barrel.

In some embodiments, a needle is coupled to a distal end of a medicament container (e.g., the needle is at the distal end of a syringe), for example, as described in U.S. Pat. Nos. 9,180,047, 9,539,139, 9,572,800, 9,636,253, 9,636,332, 9,770,361, 9,937,075, 10,555,833, and 10,517,756, which are incorporated herein by reference for all purposes. In other embodiments, the present disclosure utilizes a needle that is coupled to an actuation member inside a syringe barrel. In some embodiments, a needle disclosed herein is at least partially inside the syringe barrel. In some embodiments, prior to use, the needle neither is exposed at the distal end of the syringe barrel nor directly engages the distal end of the syringe barrel.

In some embodiments, a device disclosed herein comprises an energy storage member (e.g., one or more springs) configured to engage the needle base and the floating seal. In some embodiments, a distal end portion of the energy storage member is configured to be disposed within the syringe barrel and directly or indirectly engage the floating seal. In some embodiments, the energy storage member is configured to produce a force on a proximal end portion of the floating seal. In some embodiments, the force is sufficient to move the floating seal within the syringe barrel to convey at least a portion of a substance from the medicament container (e.g., a flowable composition lumen) via the needle when a distal tip of the needle is disposed within an apparent or potential tissue void, cavity, or vessel. Furthermore, the force is insufficient to move the floating seal within the syringe barrel when the distal tip of the needle is disposed within a tissue adjacent to (e.g., above or below) the apparent or potential tissue void, cavity, or vessel. In some embodiments, the apparent or potential tissue void, cavity, or vessel has a first density and the adjacent tissue has a second density, higher than the first density. In some embodiments, the apparent or potential tissue void, cavity, or vessel produces a first backpressure and the adjacent tissue produces a second backpressure, higher than the first backpressure.

In some embodiments, a needle is coupled to a floating seal. In other embodiments, the present disclosure utilizes a needle whose proximal end is coupled to an actuation member inside a syringe barrel, where the actuation member is separately provided and is proximal to the floating seal. In some embodiments, the proximal end of a need disclosed herein is not coupled to the floating seal. In some embodiments, prior to use, the needle can be distal to the floating seal or can be through the floating seal, but the proximal end of the needle remains distal to the floating seal and is not fixedly attached to the floating seal.

In some embodiments, a medicament container (e.g., comprising a liquid) is provided between a proximal seal and a distal seal that each can move within a syringe barrel, for example, as described in US 2020/0069883 which is incorporated herein by reference for all purposes. In those devices, a force on the proximal side of the proximal seal is transmitted through the liquid to the distal seal which is attached to a needle. Given liquids are generally incompressible, when an operator uses too much force or applies a force abruptly on the proximal seal (e.g., through a plug coupled to the proximal seal), the force will be transmitted to the needle. With the liquid providing little compressibility to buffer the impact of the force, the needle may be inserted too deeply or too abruptly, causing damage to the target tissue (e.g., suprachoroidal space) and/or surrounding tissues. Although the positions of the proximal seal and the distal seal may be observed during injection, once a force that may cause overshooting of the needle is applied, it could already to be too late to stop the movement of the needle due to lack of the ability to buffer the impact of the force.

In contrast, in other embodiments of the present disclosure, the medicament container (e.g., flowable composition lumen) is provided between a floating seal and the distal end of a syringe barrel (where the distal end does not move relative to the syringe barrel). In some embodiments, the distal end of the syringe barrel comprises a distal seal and the flowable composition lumen is provided between the floating seal and the distal seal. In some embodiments, since the needle base is elastically connected to the floating seal (and therefore the flowable composition), the elastic connection can facilitate the operator to apply the right force and buffer the impact of that force. In addition, an operator can hold the needle base still relative to the syringe barrel and observe the movement of the floating seal in order to assess the depth of needle placement. Once fluidic communication is established between the flowable composition and an apparent or potential tissue void, cavity, or vessel, and the pressure in the flowable composition is greater than that in the apparent or potential tissue void, cavity, or vessel, the floating seal can move as the flowable composition enters the tissue, while the needle and the needle base do not have to move. Thus, precise needle placement and steady injection can be achieved and chances of needle overshooting can be effectively reduced or eliminated.

In some embodiments, a device disclosed herein is provided and/or packaged as an integrated device comprising components engaging each other. In some embodiments, a device disclosed herein does not require an operator to assemble one or more of components prior to use. In some embodiments, a device disclosed herein comprises a pre-filled medicament container (e.g., flowable composition lumen) comprising a flowable composition, such as a medicament in the form of a liquid, a solution, a suspension, a gel, an oil, an ointment, an emulsion, a cream, a foam, a lotion, and/or a paste.

Flowable compositions include liquid (e.g., solution, suspension, or the like) or semi-solid compositions (e.g., gels) that are easy to manipulate and may be injected, shaped and/or molded at or near the target tissue site as it coagulates. “Flowable” includes formulations with a low viscosity or water-like consistency to those with a high viscosity, such as a viscoelastic or a paste-like material. In some embodiments, a method disclosed herein involves injecting a viscoelastic material (e.g., a viscoelastic fluid) into an eye, e.g., between the sclera and the choroid/ciliary body of the eye in order to form a suprachoroidal space containing the viscoelastic material. In some embodiments, a viscoelastic fluid is are a non-Newtonian fluid formed by a viscous component and an elastic one, such as a blend of a solvent and a polymeric material. Examples of viscoelastic materials that can be used herein include sodium hyaluronate, Provisc (1% viscous and transparent material which is a specific fraction of sodium hyaluronate), Viscoat (a dispersive viscoelastic comprising of sodium hyaluronate and chondroitin sulphate), Amvisc (a purified fraction of sodium hyaluronate), Amvisc Plus (a 1.6% sodium hyaluronate product derived from rooster combs), sodium chondroitin sulfate/sodium hyaluronate, or DisCo Visc (4% sodium chondroitin sulfate, 1.65% sodium hyaluronate).

In various embodiments, the flowability of the formulation allows it to conform to irregularities, crevices, cracks, and/or voids in the tissue site. For example, in various embodiments, the formulation may be used to fill one or more voids, expand a tissue void (e.g., an apparent tissue void), and/or create a tissue void from a potential tissue void and optionally expand the created void. In some embodiments, upon contact with an aqueous medium (e.g., body fluid, water, etc.), the flowable composition may harden to form a drug depot that controls drug release.

In some embodiments, a therapeutic agent (e.g., a drug) is added to the flowable composition. Non-limiting examples of specific drugs and classes of drugs include β-adrenoceptor antagonists (e.g., carteolol, cetamolol, betaxolol, levobunolol, metipranolol, timolol), miotics (e.g., pilocarpine, carbachol, physostigmine), sympathomimetics (e.g., adrenaline, dipivefrine), carbonic anhydrase inhibitors (e.g., acetazolamide, dorzolamide), topoisomerase inhibitors (e.g., topotecan, irinotecan, camptothecin, lamellarin D, etoposide, teniposide, doxorubicin, mitoxantrone, amsacrine), prostaglandins, anti-microbial compounds, including anti-bacterials and anti-fungals (e.g., chloramphenicol, chlortetracycline, ciprofloxacin, framycetin, fusidic acid, gentamicin, neomycin, norfloxacin, ofloxacin, polymyxin, propamidine, tetracycline, tobramycin, quinolines), anti-viral compounds (e.g., acyclovir, cidofovir, idoxuridine, interferons), aldose reductase inhibitors, anti-inflammatory and/or anti-allergy compounds (e.g., steroidal compounds such as triamcinolone, betamethasone, clobetasone, dexamethasone, fluorometholone, hydrocortisone, prednisolone and non-steroidal compounds such as antazoline, bromfenac, diclofenac, indomethacin, lodoxamide, saprofen, sodium cromoglycate), artificial tear/dry eye therapies, local anesthetics (e.g., amethocaine, lignocaine, oxbuprocaine, proxymetacaine), cyclosporine, diclofenac, urogastrone and growth factors such as epidermal growth factor, mydriatics and cycloplegics, mitomycin C, and collagenase inhibitors and treatments of age-related macular degeneration such as pegagtanib sodium, ranibizumab, aflibercept and bevacizumab.

In one embodiment, the therapeutic agent is an integrin antagonist, a selectin antagonist, an adhesion molecule antagonist (e.g., intercellular adhesion molecule (ICAM)-1, ICAM-2, ICAM-3, platelet endothelial adhesion molecule (PCAM), vascular cell adhesion molecule (VCAM)), a leukocyte adhesion-inducing cytokine or growth factor antagonist (e.g., tumor necrosis factor-α (TNF-α), interleukin-1B (IL-1B), monocyte chemotatic protein-1 (MCP-1), or a vascular endothelial growth factor (VEGF)). In some embodiments, a vascular endothelial growth factor (VEGF) inhibitor is administered with one of the microneedles described herein. In some embodiments, two drugs are delivered by the methods described herein. The compounds may be administered in one formulation, or administered serially, in two separate formulations. For example, both a VEGF inhibitor and VEGF are provided. In some embodiments, the VEGF inhibitor is an antibody, for example a humanized monoclonal antibody. In further embodiments, the VEGF antibody is bevacizumab. In another embodiment, the VEGF inhibitor is ranibizumab, aflibercept or pegaptanib. In still other embodiments, the devices and methods described herein can be used to deliver one or more of the following VEGF antagonists: AL8326, 2C3 antibody, AT001 antibody, HyBEV, bevacizumab (Avastin), ANG3070, APX003 antibody, APX004 antibody, ponatinib (AP24534), BDM-E, VGX100 antibody (VGX100 CIRCADIAN), VGX200 (c-fos induced growth factor monoclonal antibody), VGX300, COSMIX, DLX903/1008 antibody, ENMD2076, Sutent (sunitinib malate), INDUS815C, R84 antibody, KD019, NM3, allogenic mesenchymal precursor cells combined with an anti-VEGF agent or antibody, MGCD265, MG516, VEGF-Receptor kinase inhibitors, MP0260, NT503, anti-DLL4/VEGF bispecific antibody, PAN90806, Palomid 529, BD0801 antibody, XV615, lucitanib (AL3810, E3810), AMG706 (motesanib diphosphate), AAV2-sFLT01, soluble Fltl receptor, Cediranib (Recentin), AV-951 (Tivozanib, KRN-951), Stivarga (regorafenib), Volasertib (BI6727), CEP11981, KH903, Lenvatinib (E7080), terameprocol (EM1421), ranibizumab (Lucentis), Votrient (pazopanib hydrochloride), PF00337210, PRS050, SP01 (curcumin), Carboxyamidotriazole orotate, hydroxychloroquine, linifanib (ABT869, RG3635), Iluvien (fluocinolone acetonide), ALG1001, AGN150998, DARPin MP0112, AMG386, ponatinib (AP24534), AVA101, Vargatef (nintedanib), BMS690514, KH902, golvatinib (E7050), Afinitor (everolimus), Dovitinib lactate (TKI258, CHIR258), ORA101, ORA102, Axitinib (Inlyta, AG013736), Plitidepsin (Aplidin), Lenvatinib mesylate, PTC299, aflibercept (Zaltrap, Eylea), pegaptanib sodium (Macugen, LI900015), Visudyne (verteporfin), bucillamine (Rimatil, Lamin, Brimani, Lamit, Boomiq), R3 antibody, AT001/r84 antibody, troponin (BLS0597), EG3306, vatalanib (PTK787), Bmab100, GSK2136773, Anti-VEGFR Alterase, Avila, CEP7055, CLT009, ESBA903, HuMax-VEGF antibody, GW654652, HMPL010, GEM220, HYB676, JNJ17029259, TAK593, XtendVEGF antibody, Nova21012, Nova21013, CP564959, Smart Anti-VEGF antibody, AG028262, AG13958, CVX241, SU14813, PRS055, PG501, PG545, PT1101, TG100948, ICS283, XL647, enzastaurin hydrochloride (LY317615), BC194, quinolines, COT601M06.1, COT604M06.2, Mabion VEGF, SIR-Spheres coupled to anti-VEGF or VEGF-R antibody, Apatinib (YN968D1), and AL3818. In addition, delivery of a VEGF inhibitor or VEGF antagonist using the microneedle devices and methods disclosed herein may be combined with one or more agents listed herein or with other agents known in the art.

In some embodiments, one or more components of a system or device disclosed herein are configured to be assembled with one another. For example, the system or device may comprise one or more syringe barrels.

In some embodiments, the system or device may comprise two or more units, such as a first syringe unit comprising: a first syringe barrel; a needle base in the first syringe barrel; and a needle comprising a needle proximal end engaging the needle base and a needle distal end. In some embodiments, the system or device may comprise a second syringe unit configured to engage a distal end of the first syringe unit, comprising: a second syringe barrel; and a floating seal in the second syringe barrel, and when the first and second syringe units are engaged, the floating seal is configured to elastically engage the needle base. In some embodiments, the system or device may comprise a third syringe unit configured to engage a distal end of the second syringe unit, comprising a third syringe barrel enclosing a flowable composition, and the needle base can be configured to advance the needle to place the needle proximal end and/or the needle distal end in the flowable composition. In any of the embodiments herein, the system or device can comprise one or more syringe units, optionally a fourth syringe unit configured to engage a distal end of the third syringe unit.

In some embodiments, the system or device may comprise a first syringe unit comprising: a first syringe barrel; a needle base and a floating seal in the first syringe barrel elastically engaging each other, the needle base being proximal to the floating seal; and a needle comprising a needle proximal end engaging the needle base and a needle distal end, the needle comprising: (i) a needle distal opening, (ii) a needle body opening between the needle proximal end and the needle distal end, the needle body opening being proximal to the needle distal opening, and (iii) a needle body passageway connecting the needle distal opening and the needle body opening. In some embodiments, the system or device may further comprise a second syringe unit configured to engage a distal end of the first syringe unit, comprising a second syringe barrel enclosing a flowable composition, and the needle base can be configured to advance the needle to place the needle proximal end and/or the needle distal end in the flowable composition. In any of the embodiments herein, the device can comprise one or more syringe units, optionally a third syringe unit configured to engage a distal end of the second syringe unit.

In some embodiments, the system or device may comprise a first syringe unit comprising: a first syringe barrel; a needle base in the first syringe barrel; and a needle comprising a needle proximal end engaging the needle base and a needle distal end, the needle comprising: (i) a needle distal opening, (ii) a needle body opening between the needle proximal end and the needle distal end, the needle body opening being proximal to the needle distal opening, and (iii) a needle body passageway connecting the needle distal opening and the needle body opening. In some embodiments, the system or device may further comprise a second syringe unit configured to engage a distal end of the first syringe unit, comprising: a second syringe barrel; a floating seal in the second syringe barrel, and when the first and second syringe units are engaged, the floating seal is configured to elastically engage the needle base; and a flowable composition, and the needle base can be configured to advance the needle to place the needle proximal end and/or the needle distal end in the flowable composition. In any of the embodiments herein, the device can comprise one or more syringe units, optionally a third syringe unit configured to engage a distal end of the second syringe unit.

In some embodiments, the present disclosure provides in a medical puncturing device comprising: a syringe barrel, wherein the syringe barrel comprises a distal closed end and a proximal open end; an actuation unit (e.g., an elastic movement unit) comprising an actuation member (e.g., pressing element) and a floating seal, wherein the floating seal is positioned inside the syringe barrel and can elastically engage with the actuation member (e.g., pressing element); a hollow puncture needle attached to the actuation member (e.g., pressing element), wherein the hollow puncture needle comprises a needle distal opening and a needle body opening, and wherein the needle body opening is proximal to the floating seal (the needle distal opening can be proximal to the floating seal, e.g., the entire length of the needle is proximal to the floating seal, or alternatively, the needle can be through the floating seal such that the needle distal opening is distal to the floating seal); and a flowable composition lumen (e.g., for a fluid or gel), wherein the flowable composition lumen is formed by the syringe barrel distal closed end, a syringe barrel lumen wall (e.g., a portion of the syringe barrel), and the floating seal.

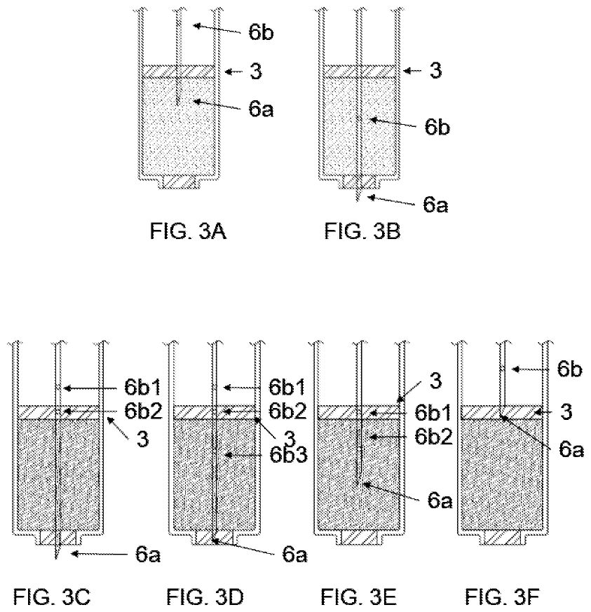

In some embodiments, the medical puncturing device is configured such that the hollow puncture needle can be moved forward by pressing the actuation member (e.g., pressing element). In some embodiments, the hollow puncture needle sequentially pierces the floating seal and the syringe barrel distal closed end, thus connecting the flowable composition lumen, the needle body opening, and the needle distal opening. In some embodiments, the hollow puncture needle is pre-inserted into the floating seal. For example, the needle distal opening can be in the floating seal and blocked by the floating seal, and the needle can be advanced through the flowable composition lumen to pierce the syringe barrel distal closed end. In some embodiments, the hollow puncture needle is pre-inserted through the floating seal. For example, the needle distal opening can be in the flowable composition lumen, while the needle body opening is proximal to the floating seal or in the floating seal (e.g., the needle body opening can be blocked by the floating seal as shown in FIG. 3E), and then the needle can be advanced to pierce the syringe barrel distal closed end. In some embodiments, the hollow puncture needle is pre-inserted through the floating seal and in or through the syringe barrel distal closed end. For example, the needle distal opening can be in a distal seal at the syringe barrel distal closed end (e.g., the needle distal opening can be blocked by the distal seal) or distal to the distal seal and/or the syringe barrel distal closed end, while the needle body opening is proximal to the floating seal (e.g., as shown in FIG. 3D, 6b1), in the floating seal (e.g., the needle body opening can be blocked by the floating seal as shown in FIG. 3D, 6b2), or in the flowable composition lumen (e.g., as shown in FIG. 3D, 6b3), and then the needle can be advanced through the syringe barrel distal closed end and exposing the needle distal opening for puncturing a tissue.

Optionally, the medical puncturing device comprises a state wherein the flowable composition lumen, the needle body opening, and the needle distal opening are in fluidic communication. For example, in a fluidic communication state, the needle body opening can be proximal to the floating seal, while the needle distal opening is distal to the floating seal and in the flowable composition lumen. In the fluidic communication state, the needle and/or the floating seal can be moved. For example, the floating seal can be moved under the elastic resilience between the floating seal and the actuation member (e.g., pressing element) such as that the floating seal seals or blocks the needle body opening, thereby preventing or terminating discharge of the flowable composition (such as a gel) from the needle body opening and/or from the needle distal opening.

Optionally, in the fluidic communication state, the floating seal can seal the needle body opening when it moves forward and contacts the syringe barrel distal closed end, thereby preventing or terminating discharge of the flowable composition (such as a gel) from the needle body opening and/or from the needle distal opening.

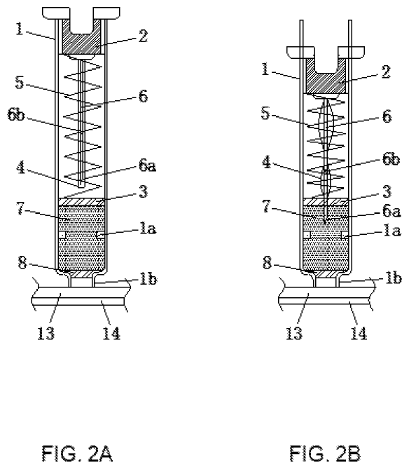

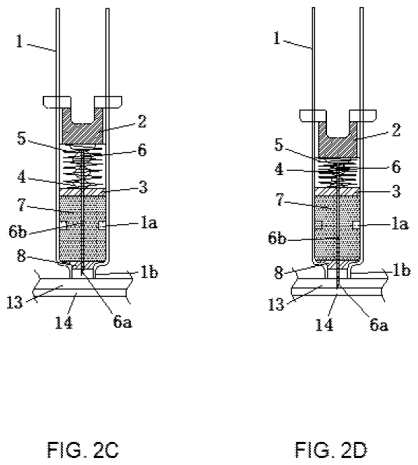

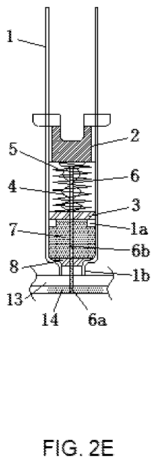

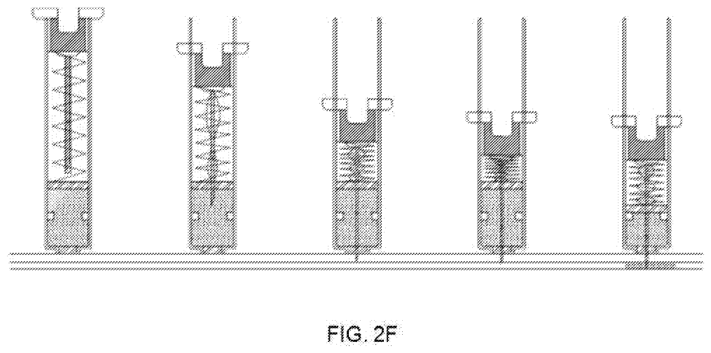

Optionally, a stopper such as an axial stopper can be provided inside the syringe lumen, distal to the floating seal. In some embodiments, the stopper can be used to limit the forward movement of the floating seal. In some embodiments, the medical puncturing device comprises a fluidic communication state, wherein the flowable composition lumen is connected to the needle body opening and the needle distal opening. When the medical puncturing device is in the fluidic communication state, the needle body opening can be at the distal end of the stopper (e.g., as shown in FIG. 2D), and the floating seal can move forward due to the elastic engagement with the actuation member (e.g., pressing element).

Optionally, the medical puncturing device comprises a manual control element, which is attached to the floating seal and is extended outside of the syringe barrel.

Optionally, the medical puncturing device comprises a pre-puncture state after the hollow puncture needle pierces the syringe barrel distal closed end, a surface tissue puncture state, and a fluidic communication state after the puncture. In the pre-puncture state, the surface tissue puncture state, and the fluidic communication state, the length range of the hollow puncture needle extended outside of the syringe barrel distal closed end can correspond to a pre-puncture length range, a surface tissue puncture length range, and a fluidic communication length range, respectively, wherein: when the length of the of the hollow puncture needle extended outside of the syringe barrel distal closed end is within the pre-puncture length range, the needle body opening remains above the flowable composition lumen (e.g., the needle body opening can be proximal to and within the floating seal); and/or when the length of the of the hollow puncture needle extended outside of the syringe barrel distal closed end is within the surface tissue puncture length range, at least part of the needle body opening is connected to the flowable composition lumen; and/or, when the length of the of the hollow puncture needle extended outside of the syringe barrel distal closed end is within the fluidic communication length range, the needle body opening is positioned within the flowable composition lumen.

Optionally, an axially extended circular contacting element is formed at the syringe barrel distal closed end, wherein the difference between the upper and lower limits of the pre-puncture length range equals to the axial length of the circular contacting element.

Optionally, the elastic movement unit comprises a elastic sheath covering the outside of the hollow puncture needle. When the needle body opening is proximal to the floating seal, the elastic sheath can seal the needle body opening. In some embodiments, when the flowable composition is a gel, it may not be necessary to seal the needle body opening when it is proximal to the floating seal.

Optionally, the medical puncturing device comprises a catheter guiding structure which is used to thread the catheter into a cavity (e.g., a needle body passageway connected to the needle distal opening and/or the needle body opening) of the hollow puncture needle.

Optionally, the catheter guiding structure comprises an angled guiding groove which is formed on the floating seal and extends towards the hollow puncture needle in an angle.

Optionally, the angled guiding groove is set to be through the floating seal in the front and back direction. In some embodiments, the catheter guiding structure further comprises a one-way valve which is embedded in the angled guiding groove and can be opened and closed, and/or a guiding groove plug inserted in the angled guiding groove.

Optionally, the angled guiding groove is set to be on the upper surface of the floating seal and is a non-through groove.

Optionally, the needle body opening is formed as an angled opening which opens obliquely backwards.

Optionally, the catheter guiding structure comprises an angled guiding needle hole formed on the body wall of the hollow puncture needle and opens obliquely backwards. In some embodiments, the medical puncturing device comprises a fluidic communication state wherein the flowable composition lumen is in connection with the needle body opening and the needle distal opening. In the fluidic communication state, the angled guiding needle hole is positioned proximal to the floating seal.

Optionally, the catheter guiding structure further comprises a one-way valve which is embedded in the angled guiding needle hole and can be opened and closed, or a guiding groove plug inserted in the angled guiding needle hole.

Optionally, the catheter guiding structure comprises a puncturable central guiding groove that is formed on the center of the proximal surface of the actuation member (e.g., pressing element). In some embodiments, a needle proximal opening is formed on the hollow puncture needle and the needle proximal opening is set to axially align with the central guiding groove.

Optionally, the medical puncturing device comprises a puncture control module and a fluid storage module that are independently manufactured and formed, wherein: the puncture control module comprises a first syringe unit and the elastic movement unit and the hollow puncture needle provided inside the first syringe unit; the fluid storage module comprises a second syringe unit, the flowable composition lumen formed inside the barrel of the second syringe unit, and a module packaging component which is removably packaged to the proximal end of the second syringe unit; and a removable connection structure is formed between the first syringe unit and the second syringe unit.

In a second aspect, the present disclosure provides a medical apparatus assembly. In some embodiments, the medical apparatus assembly comprises a catheter and the medical puncturing device comprising a catheter guiding structure.

Optionally, the medical apparatus assembly further comprises a hollow auxiliary guiding needle which is matched to use with the catheter guiding structure. In some embodiments, when the auxiliary guiding needle is connected to the catheter guiding structure, the catheter can sequentially go through the needle body passageway of the auxiliary guiding needle and the catheter guiding structure and be threaded into the needle body passageway of the hollow puncture needle.

In some embodiments, when using the medical puncturing device of the present disclosure, a user can first apply pressure to the actuation member (e.g., pressing element) to drive the hollow puncture needle sequentially through the floating seal and the syringe barrel distal closed end. When the needle distal opening of the hollow puncture needle reaches apparent or potential tissue gaps, cavity systems, and vessels, the needle body opening has already been positioned in the flowable composition lumen, and the floating seal has already formed an elastic engagement with the actuation member (e.g., pressing element). In some embodiments, the fluid pressure in the flowable composition lumen can be made higher than the pressure inside the an apparent or potential tissue void, cavity, or vessel.

At this time, the fluid inside the flowable composition lumen can flow into the an apparent or potential tissue void, cavity, or vessel through the needle body opening and the needle distal opening. During the injection process, just by maintaining the position of the actuation member (e.g., pressing element), under the action of the elastic engagement between the floating seal and the actuation member (e.g., pressing element), the fluid inside the flowable composition lumen can flow into the needle body opening (and then through the needle body passageway and out of the needle distal opening), thereby achieving injection, penetration, and/or expansion of the an apparent or potential tissue void, cavity, or vessel. Additionally, the medical apparatus assembly as describe in the present disclosure can achieve implantation of catheter and other medical device through the medical puncturing device, e.g., through a catheter guiding structure and a cavity of the needle described herein.

In some embodiments, before the hollow puncture needle pierces into an apparent or potential tissue void, cavity, or vessel, the external pressure on the needle distal opening is higher than the fluid pressure in the flowable composition lumen, thus fluid cannot flow out of the needle distal opening. Thus, by observing whether the floating seal moves forward due to the elastic engagement with the actuation member (e.g., pressing element), it is possible to determine whether the hollow puncture needle has already pierced into an apparent or potential tissue void, cavity, or vessel, thereby reminding the operator of the current punctuation depth to ensure accurate puncture. Since the injection is controlled by fluid pressure changes in the flowable composition lumen, the injection process does not require an operator to manually apply thrust or force during the injection process, thus fluctuations in the flow speed can be prevented and stable injection can be achieved.

### III. Methods for Medical Penetration

In some embodiments, described herein are methods for medical puncture, for example, in an eye or other organs or tissues.

As shown in FIGS. 1-11B, in some embodiments the present disclosure provides a medical puncturing or penetration device which comprises syringe barrel 1, an actuation unit (e.g., an elastic movement unit for pushing a needle), hollow puncture needle 6, and flowable composition lumen 7.

In some embodiments, syringe barrel 1 comprises a distal closed end and a proximal open end. In some embodiments, syringe barrel 1 can be designed to have two open ends in an axial direction, and sealing of the distal end can be achieved by installing distal seal 8 at the distal opening of syringe barrel 1. In some embodiments, distal seal 8 can be made of a material that can be punctured by hollow puncture needle 6, such as rubber or the like.

In some embodiments, the actuation unit (e.g., elastic movement unit) comprises actuation member (e.g., pressing element) 2 and floating seal 3, where the floating seal 3 sealingly engages an inside wall of the syringe barrel and is configured to move in an axial direction, e.g., toward the distal end or the proximal end of the syringe barrel. In some embodiments, actuation member (e.g., pressing element) 2 or a portion thereof is located outside the proximal opening of the syringe barrel, so that an operator can press on the actuation member (e.g., pressing element) or portion thereof manually. In some embodiments, floating seal 3 elastically engages actuation member 2, and when pressure is applied on actuation member 2, floating seal 3 can move forward or backward relative to the actuation member (e.g., pressing element). In some embodiments, floating seal 3 is configured to move toward the distal end of the syringe barrel. In some embodiments, floating seal 3 is configured to move toward the proximal end of the syringe barrel. In some embodiments, the position of the actuation member (e.g., pressing element) relative to the syringe barrel is kept still, floating seal 3 is configured to move forward (e.g., in a distal direction) under elastic resilience due to the elastic engagement with the actuation member (e.g., pressing element).

In some embodiments, hollow puncture needle 6 is fixedly connected to actuation member 2. When no pressure is applied to actuation member 2, hollow puncture needle 6 remains proximal to floating seal 3 and the two do not come into contact. In some embodiments, hollow puncture needle 6 itself comprises needle distal opening 6a and needle body opening 6b. In some embodiments, needle distal opening 6a and needle body opening 6b are connected through a needle cavity or needle body passageway of hollow puncture needle 6.

In some embodiments, flowable composition lumen 7 is used for storage, e.g., of a medication and other flowable composition such as a liquid or a gel. In some embodiments, the flowable composition lumen is enclosed by a distal closed end of the syringe barrel, a lumen wall of the syringe barrel, and floating seal 3; that is, the flowable composition lumen occupies a distal portion of a syringe barrel lumen. In some embodiments, since floating seal 3 can move along in an axial direction, flowable composition lumen 7 is configured to have a variable volume, thus the fluid pressure inside flowable composition lumen 7 can change due to an axial movement of floating seal 3.

In some embodiments, using a medical puncturing device disclosed herein comprises applying pressure on actuation member 2, thereby advancing hollow puncture needle 6 forward in a distal direction, sequentially through floating seal 3 (e.g., by puncturing the floating seal or forcing open an existing aperture or slit through the floating seal) and through a distal closed end (e.g., by puncturing the distal closed end or forcing open an existing aperture or slit through the distal closed end) of the syringe barrel. The existing aperture or slit may be through the floating seal, e.g., from a proximal surface of the floating seal to a distal surface of the floating seal, thereby providing a through hole in the floating seal. The existing aperture or slit may be not through the entire floating seal, and advancing the needle distal end through the floating seal may comprise advancement through the existing aperture or slit and puncturing a portion of the floating seal in any suitable combination. For instance, the needle distal end may first advance through an existing aperture or slit from a proximal surface and then puncture the floating seal before emerging from a distal surface of the floating seal, or vice versa. In some embodiments, hollow puncture needle 6 pierces into an apparent or potential tissue void, cavity, or vessel, thereby placing needle distal opening 6a in the apparent or potential tissue void, cavity, or vessel. In some embodiments, needle body opening 6b is positioned inside flowable composition lumen 7, and floating seal 3 is elastically engaged with actuation member 2. In some embodiments, the fluid pressure in flowable composition lumen 7 is higher than the pressure inside the apparent or potential tissue void, cavity, or vessel.

At this time, the flowable composition inside flowable composition lumen 7 can flow through needle body opening 6b and needle distal opening 6a and into the apparent or potential tissue void, cavity, or vessel. In some embodiments, during an injection process, a user can simply maintain the pressure on actuation member 2, e.g., without further increasing the pressure. Under the action of the elastic engagement between floating seal 3 and actuation member 2, the flowable composition (e.g., a solution, a suspension, or a gel) inside flowable composition lumen 7 can enter needle body opening 6b and through the needle body passageway, thus achieving injection, penetration, and/or expansion of the apparent or potential tissue void, cavity, or vessel.

In some embodiments, before hollow puncture needle 6 pierces into an apparent or potential tissue void, cavity, or vessel, external pressure on needle distal opening 6a is higher than the fluid pressure in flowable composition lumen 7, e.g., due to the needle distal opening being in a tissue denser, harder, and/or less deformable than the apparent or potential tissue void, cavity, or vessel. Thus, the flowable composition inside the flowable composition lumen cannot exist needle distal opening 6a and into the surrounding tissue. Take the puncture process of the SCS of the eye as an example, when hollow puncture needle 6 has already pierced sclera 13 but has not yet pierced SCS 14, regardless of whether needle body opening 6b is in fluid communication with flowable composition lumen 7 or not, the flowable composition would not exit from needle distal opening 6a. This is because sclera 13 is relatively dense, and when needle distal opening 6a is inside sclera 13, a relatively high external pressure is applied on needle distal opening 6a. The external pressure is higher than the fluid pressure in flowable composition lumen 7, and the dense tissue such as the sclera essentially functions as a plug that prevents the flowable composition from flowing out.

In some embodiments, by observing whether floating seal 3 moves forward due to the elastic engagement when actuation member 2 is held still under pressure, an operator can determine whether hollow puncture needle 6 has already pierced into an apparent or potential tissue void, cavity, or vessel, thereby informing the operator of the current needle depth and/or location of the needle distal opening and ensure accurate needle placement. In some embodiments, since the injection is controlled by fluid pressure changes in flowable composition lumen 7, the injection process does not require manually applying a force that is transmitted via relatively rigid medium (e.g., solid or liquid) in order to advance and precisely place the needle tip into an apparent or potential tissue void, cavity, or vessel. Rather, an abrupt force applied to actuation member 2 can be buffered due to the elastic engagement between actuation member 2 and floating seal 3, thus allowing more controllable and steady movement of the floating seal. In some embodiments, using a device disclosed herein, fluctuations in the flow speed can be prevented or reduced and steady injection can be achieved.

In some embodiments, when hollow puncture needle 6 pierces through the syringe barrel distal closed end, the medical puncturing device can be in at least three states: a pre-puncture state, a surface tissue puncture state, and a fluidic communication state.

In some embodiments, in the pre-puncture state, the length range of hollow puncture needle 6 extending from the syringe barrel distal closed end is a pre-puncture length range. Within this range, hollow puncture needle 6 has not yet started puncturing an organism or a tissue thereof.

In some embodiments, a system or device of the present disclosure comprises a flowable composition lumen pre-filled with a flowable composition. In some embodiments, prior to use of the system or device, the needle is already through the floating seal. In some embodiments, prior to use of the system or device, the needle is already through the floating seal and the syringe barrel distal end, e.g., a distal seal sealing the syringe barrel distal end.

In some embodiments, the flowable composition is of a relatively high viscosity, e.g., higher than water-like consistency, such as a gel or paste-like material. Elastic sleeve or sheath 4 shown in the figures of the present disclosure is optional, especially when the viscosity of the flowable composition is sufficient to prevent discharge from the needle body opening and/or needle distal opening when the openings are in the flowable composition lumen. For example, as shown in FIG. 3A, the needle can be through the floating seal such that needle body opening 6b is proximal to the floating seal while needle distal opening 6a is in the flowable composition lumen. Discharge of the flowable composition from the needle body opening can be prevented due to viscosity of the composition, and the elastic sheath is optional. Alternatively, as shown in FIG. 3B, the needle body opening 6b can be in the flowable composition lumen while needle distal opening 6a is outside the flowable composition lumen. Discharge of the flowable composition from the needle distal opening can be prevented due to viscosity of the composition, until the needle distal opening reaches a target tissue, such as an apparent or potential tissue void, cavity, or vessel.

In some embodiments, for example prior to or during the use of the system or device, needle distal opening 6a can be outside the flowable composition lumen, while needle body opening 6b can be proximal to the floating seal (e.g., as shown in FIG. 3C, 6b1) or within the floating seal (e.g., as shown in FIG. 3C, 6b2). Discharge of the flowable composition from the needle distal opening can be prevented due to viscosity of the composition, until the needle distal opening reaches a target tissue, such as an apparent or potential tissue void, cavity, or vessel.

In some embodiments, for example prior to or during the use of the system or device, needle distal opening 6a can be within a distal seal at the syringe barrel distal closed end (e.g., the needle distal opening can be blocked by the distal seal), while needle body opening 6b can be proximal to the floating seal (e.g., as shown in FIG. 3D, 6b1), within the floating seal (e.g., as shown in FIG. 3D, 6b2), or within the flowable composition lumen (e.g., as shown in FIG. 3D, 6b3). Discharge of the flowable composition from the needle distal opening and the needle body opening can be prevented.

In some embodiments, for example prior to or during the use of the system or device, needle distal opening 6a can be within the flowable composition lumen, while needle body opening 6b can be within the floating seal (e.g., as shown in FIG. 3E, 6b1) or within the flowable composition lumen (e.g., as shown in FIG. 3E, 6b2). Discharge of the flowable composition from the needle body opening can be prevented.

In some embodiments, for example prior to or during the use of the system or device, needle distal opening 6a can be within the floating seal, while needle body opening 6b can be proximal to the floating seal (e.g., as shown in FIG. 3F, 6b). Discharge of the flowable composition from the needle body opening can be prevented.

In some embodiments, in the surface tissue puncture state, the length range of hollow puncture needle 6 extending from the syringe barrel distal closed end is a surface tissue puncture length range. Within this range, the distal end of hollow puncture needle 6 has entered a surface tissue (for example, pierced into sclera 13) but has not yet entered the apparent or potential tissue void, cavity, or vessel (for example, not pierced into SCS 14). In some embodiments, because the surface tissue is relatively dense, external pressure on needle distal opening 6a is higher than the fluid pressure in flowable composition lumen 7, therefore, no matter whether needle body opening 6b is connected to flowable composition lumen 7 or not, the flowable composition does not enter needle body opening 6b and/or exit needle distal opening 6a.

In some embodiments, while in the fluidic communication state, the length range of hollow puncture needle 6 extending from the syringe barrel distal closed end is the a fluidic communication. Within this range, the distal end of hollow puncture needle 6 has pierced into the apparent or potential tissue void, cavity, or vessel. In some embodiments, the device can be designed such that in the fluidic communication state, the fluid pressure in flowable composition lumen 7 is higher than the pressure inside the apparent or potential tissue void, cavity, or vessel. In some embodiments, in the fluidic communication state, needle body opening 6b has already positioned inside flowable composition lumen 7, and due to a difference in the internal (e.g., in the apparent or potential tissue void, cavity, or vessel) and external (e.g., in flowable composition lumen 7) pressures, the flowable composition inside lumen 7 can flow into the apparent or potential tissue void, cavity, or vessel through needle body opening 6b, the needle body passageway, and then needle distal opening 6a.

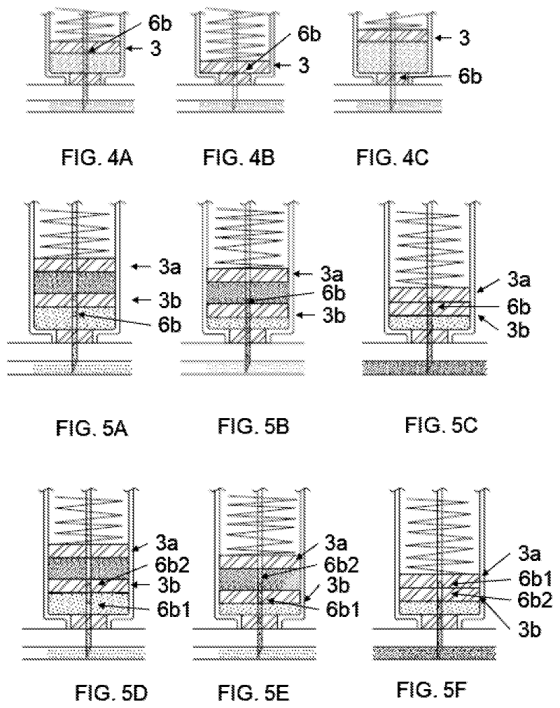

In some embodiments, floating seal 3 moves distally due to the elastic engagement with actuation member 2 (e.g., due to the pressure in the flowable composition lumen being higher than a backpressure at the needle distal opening in the apparent or potential tissue void, cavity, or vessel) until the floating seal seals needle body opening 6b (e.g., as shown in FIGS. 4A-4B). In some embodiments, the axial dimension of the needle body opening is no greater than the thickness of the floating seal. In some embodiments, the needle body opening can be completely sealed or blocked by the floating seal, at which time no more flowable composition exits needle distal opening 6a to enter the tissue void. In some embodiments, when the floating seal blocks the needle body opening, only a portion of the total volume of flowable composition has exited needle distal opening 6a (e.g., as shown in FIG. 4A). In some embodiments, when the floating seal blocks the needle body opening, the total volume of flowable composition in the lumen has exited needle distal opening 6a (e.g., as shown in FIG. 4B).

In some embodiments, the needle body opening can be in the distal seal or in a tissue of a subject, the flowable composition will stop existing needle distal opening 6a (e.g., as shown in FIG. 4C). In some embodiments, the distance between needle distal opening 6a and needle body opening 6b can be keep constant. In some embodiments, the distance between needle distal opening 6a and needle body opening 6b can be varied. For example, a needle having a suitable distance between needle distal opening 6a and needle body opening 6b can be selected based on a known or estimated depth of the tissue to be accessed. In some embodiments, stopper 1a is provided inside the syringe lumen and can be used to limit the forward movement of floating seal 3 in order to achieve precise injection, for example, injection of a pre-determined volume.

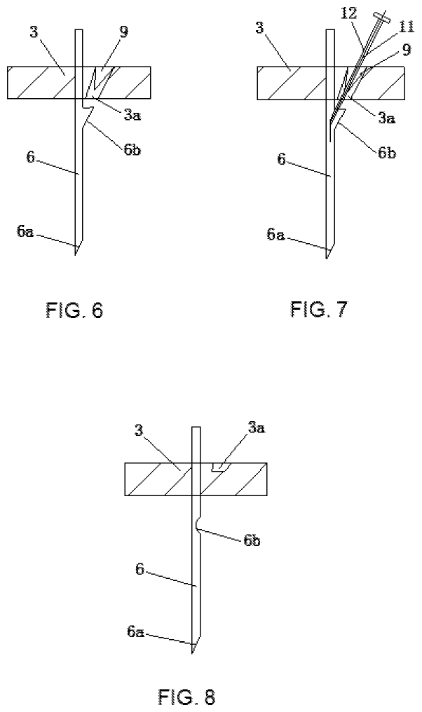

In some embodiments, once floating seal 3 contacts stopper 1a, further distal movement of the floating seal is limited, thereby stabilizing floating seal 3 for subsequent operation, for example, as shown in FIGS. 6-11B.

In some embodiments, a system or device disclosed herein comprises two or more floating seals. For example, as shown in FIG. 5A, a first lumen is formed between floating seal 3b and the distal seal of the syringe barrel, and a second lumen is formed between floating seal 3a and floating seal 3b. In some embodiments, the first lumen and the second lumen comprise the same flowable material. In some embodiments, the first lumen and the second lumen comprise different flowable compositions. In some embodiments, the first lumen and the second lumen comprise the same medicament (e.g., active pharmaceutical ingredient) in the same or different flowable carriers or excipients. In some embodiments, the first lumen and the second lumen comprise different medicaments (e.g., active pharmaceutical ingredients) in the same or different flowable carriers or excipients. In some embodiments, the first lumen comprises a medicament and the second lumen comprises a pharmaceutically acceptable carrier or excipient such as a saline, or vice versa.

In some embodiments, the flowable compositions in the first lumen and the second lumen can be sequentially delivered to an apparent or potential tissue void, cavity, or vessel. In some embodiments, the flowable compositions in the first lumen and the second lumen can be mixed in the apparent or potential tissue void, cavity, or vessel. In some embodiments, the flowable composition in the first lumen enters the apparent or potential tissue void, cavity, or vessel in order to access and/or expand the tissue void, cavity, or vessel. Subsequently, the flowable composition in the second lumen comprising a medicament can enter the apparent or potential tissue void, cavity, or vessel. For example, as shown in FIG. 5A, when needle distal opening 6a is in the apparent or potential tissue void, cavity, or vessel while needle body opening 6b is in the first lumen (between floating seal 3b and the distal seal of the syringe barrel), the flowable composition in the first lumen is delivered to the tissue. In FIG. 5B, needle distal opening 6a can be held still in the apparent or potential tissue void, cavity, or vessel, when floating seal 3b moves distally and needle body opening 6b contacts the second lumen (between floating seal 3a and floating seal 3b). This way, the flowable composition in the second lumen starts to be delivered to the tissue until a volume is delivered and/or floating seal 3a (or floating seal 3a and floating seal 3b together) blocks needle body opening 6b, as shown in FIG. 5C. In some embodiments, a set (e.g., predetermined) volume of the flowable composition in the first lumen and/or a set (e.g., predetermined) volume of the flowable composition in the second lumen can be delivered to the apparent or potential tissue void, cavity, or vessel. In some embodiments, the dimension of needle body opening 6b along the needle axis is greater than the thickness of floating seal 3b such that a first flowable composition (between floating seal 3b and the distal seal of the syringe barrel) and a second flowable composition (between floating seal 3b and floating seal 3a) can be sequentially and continuously delivered to the apparent or potential tissue void, cavity, or vessel through the needle distal opening. In some embodiments, the dimension of needle body opening 6b along the needle axis is no greater than the thickness of floating seal 3a and floating seal 3b combined. In some embodiments, the dimension of needle body opening 6b along the needle axis is greater than the thickness of floating seal 3b and less than the thickness of floating seal 3a and floating seal 3b combined. In some embodiments, a system or device disclosed herein comprises one or more additional floating seals (e.g., a third floating seal, 3c) that are proximal to floating seal 3a, distal to floating seal 3b, and/or between floating seal 3a and floating seal 3b, such that a third flowable composition may be delivered before the first flowable composition, after the second flowable composition, or between the first and second flowable compositions.

In some embodiments, a system or device disclosed herein comprises two or more needle body openings. In some embodiments, a system or device disclosed herein comprises two or more needle body openings and two or more floating seals. For example, as shown in FIG. 5D, when needle distal opening 6a is in the apparent or potential tissue void, cavity, or vessel while needle body opening 6b1 is in the first lumen (between floating seal 3b and the distal seal of the syringe barrel) and needle body opening 6b2 is blocked by floating seal 3b, the flowable composition in the first lumen is delivered to the tissue. In FIG. 5E, needle distal opening 6a can be held still in the apparent or potential tissue void, cavity, or vessel, when floating seal 3b moves distally to block needle body opening 6b1, allowing needle body opening 6b2 to contact the second lumen (between floating seal 3a and floating seal 3b). This way, the flowable composition in the second lumen starts to be delivered to the tissue until a volume is delivered and/or floating seal 3a (or floating seal 3a and floating seal 3b together) blocks needle body opening 6b2 (and/or needle body opening 6b1) as shown in FIG. 5F. In some embodiments, a set (e.g., predetermined) volume of the flowable composition in the first lumen and/or a set (e.g., predetermined) volume of the flowable composition in the second lumen can be delivered to the apparent or potential tissue void, cavity, or vessel. In some embodiments, the distance between needle body opening 6b1 and needle body opening 6b2 along the needle axis is greater than the thickness of floating seal 3b such that a first flowable composition (between floating seal 3b and the distal seal of the syringe barrel) and a second flowable composition (between floating seal 3b and floating seal 3a) can be sequentially and continuously delivered to the apparent or potential tissue void, cavity, or vessel through the needle distal opening. In some embodiments, the distance between needle body opening 6b1 and needle body opening 6b2 along the needle axis is no greater than the thickness of floating seal 3a and floating seal 3b combined. In some embodiments, the distance between needle body opening 6b1 and needle body opening 6b2 along the needle axis is greater than the thickness of floating seal 3b and less than the thickness of floating seal 3a and floating seal 3b combined. In some embodiments, a system or device disclosed herein comprises one or more additional needle body openings (e.g., a third needle body opening, 6b3) that are proximal to needle body opening 6b2, distal to needle body opening 6b1, and/or between needle body openings 6b1 and 6b2, such that a third flowable composition may be delivered before the first flowable composition, after the second flowable composition, or between the first and second flowable compositions.

Described below are multiple embodiments to control the termination of the injection process using a medical puncturing device disclosed herein.

In some embodiments, when the medical puncturing device is in a fluidic communication state, floating seal 3 moves forward due to the elastic engagement with actuation member 2 until it seals needle body opening 6b. Once needle body opening 6b is sealed, the injection process is terminated. In some embodiments, the axial position of needle body opening 6b within the flowable composition lumen 7 limits the maximum injection volume of the medical puncturing device. In some embodiments, when needle body opening 6b is blocked or sealed by floating seal 3, floating seal 3 has not contacted a wall at the syringe barrel distal closed end. In some embodiments, flowable composition lumen 7 is not completely emptied and there is still flowable composition between floating seal 3 and the wall at the syringe barrel distal closed end.

In some embodiments, when flowable composition lumen 7 needs to be emptied, floating seal 3 can be designed to seal needle body opening 6b when the floating seal contacts the syringe barrel distal closed end. In some embodiments, needle body opening 6b is at the distal end of flowable composition lumen 7. In some embodiments, floating seal 3 contacts a wall at the syringe barrel distal closed end and needle body opening 6b is blocked or sealed by floating seal 3 and/or the wall at the syringe barrel distal closed end. In some embodiments, flowable composition lumen 7 is emptied and there is no or little flowable composition between floating seal 3 and the wall at the syringe barrel distal closed end.

In some embodiments, as the flowable composition inside flowable composition lumen 7 gradually enters the apparent or potential tissue void, cavity, or vessel, there can be a state wherein the fluid pressure inside flowable composition lumen 7 reaches equilibrium with the pressure in the apparent or potential tissue void, cavity, or vessel. At this time, floating seal 3 no longer moves, due to the balance of forces. In order to continue injection and/or empty flowable composition lumen 7, additional force is needed on floating seal 3 in order to move it forward toward the syringe barrel distal closed end.

For example, as shown in FIGS. 2A-2E, one, two, or more axially extending sliding grooves (not shown) can be provided on a body wall of syringe barrel 1. A slider matching a sliding groove can be provided on actuation member 2 (e.g., a slider can comprise a portion of actuation member 2 extending outside of syringe barrel 1), thus increasing the upper limit of the movement distance or stroke of actuation member 2 since the movement is not limited by the proximal end of actuation member 2. When floating seal 3 can no longer move due to the equilibrium of forces (e.g., between pressure inside flowable composition lumen 7 and the apparent or potential tissue void, cavity, or vessel), more pressure can be applied on a slider of actuation member 2 to drive actuation member 2 forward distally, which in turn can increase the elastic resilience between floating seal 3 and actuation member 2, thus breaking the force equilibrium and moving floating seal 3 forward toward the distal end of the syringe barrel. This way, more flowable composition can be expelled from flowable composition lumen 7, in some embodiments emptying flowable composition lumen 7.

In some embodiments, other drive structures can be used to move floating seal 3 further until it contacts a wall of the syringe barrel distal closed end. Exemplary drive structures are described below.

In some embodiments, the medical puncturing device comprises an element configured for an operator to manually control movement of the floating seal using one or both hands. In some embodiment, the manual control element can be moved using one or more fingers, for example, one finger of the same hand holding the syringe barrel. In some embodiments, the manual control element is fixed to floating seal 3 and partially extends outside the syringe barrel. In some embodiments, when the flowable composition volume injected into the apparent or potential tissue void, cavity, or vessel does not reach a target volume, while floating seal 3 is no longer moving due to the equilibrium of forces, the operator can drive further movement of floating seal 3 forward by moving the portion of the manual control element that extends outside the syringe barrel, until the expelled flowable composition volume reaches the target volume. In some embodiments, using the manual control element helps empty flowable composition lumen 7. These embodiments are not limited to situations where flowable composition lumen 7 needs to be emptied.

In some embodiments, the medical puncturing device can achieve delivery (e.g., via injection) of a flowable composition of a defined volume with precision, and/or the ability to control the volume to be delivered. In some embodiments, the defined volume is a preset volume prior to the delivery. In some embodiments, the defined volume is one of multiple volumes that an operator can select during the delivery, and the delivered volume may be different from a preset volume. In some embodiments, as shown in FIGS. 1A-1E, FIGS. 2A-2E, and FIGS. 11A-11B, axial stopper 1a is provided inside the syringe lumen and distal to floating seal 3, and is used to limit the forward movement of floating seal 3. In some embodiments, when the medical puncturing device is in the fluidic communication state, needle body opening 6b can be distal to axial stopper 1a, and floating seal 3 can move forward due to the elastic engagement with actuation member 2.

In some embodiments, floating seal 3 is moved to the position limited by axial stopper 1α. In some embodiments, when floating seal 3 moves to the position limited by axial stopper 1a, pressure in flowable composition lumen 7 is still no less than the pressure inside the apparent or potential tissue void, cavity, or vessel. In some embodiments, floating seal 3 can be pushed forward to the position limited by axial stopper 1a by the elastic resilience between floating seal 3 and actuation member 2, and there is no need to rely on additional driving structure or force to move floating seal 3 to the position limited by axial stopper 1a.

In some embodiments, before floating seal 3 is moved to the position limited by axial stopper 1a by the elastic resilience between the floating seal and actuation member 2, pressure in flowable composition lumen 7 has already become equal with the pressure inside the apparent or potential tissue void, cavity, or vessel (that is, due to balance of forces, floating seal 3 is no longer moving before it reaches axial stopper 1a). At this time, just by the elastic resilience between floating seal 3 and actuation member 2, floating seal 3 is not pushed forward to the position limited by axial stopper 1a. Thus, in some embodiments, one or more additional driving structure or mechanism can be employed to further push forward floating seal 3. For example, the additional driving structure or mechanism can comprise a manual control element described herein (e.g., as shown in FIGS. 2A-2E). In some embodiments, axial stopper 1a provides a mechanism for achieving fluid injection of set volumes.

Described below are multiple embodiments for puncture and injection timing of a medical puncturing device disclosed herein.

In some embodiments, when the medical puncturing device is in pre-puncture state, that is, when the length of hollow puncture needle 6 extending from the syringe barrel distal closed end is within the pre-puncture length range (or when hollow puncture needle 6 has already pierced the syringe barrel distal closed end but has not yet started puncturing the organism or a tissue thereof), needle body opening 6b remains above (e.g., proximal to) flowable composition lumen 7. When provided in this way, early leakage from needle distal opening 6a can be prevented and the reliability of the medical puncturing device can be improved.

In some embodiments, corresponding structure(s) can be provided on the device to prevent early leakage before hollow puncture needle 6 punctures the tissue and/or before needle distal opening 6a reaches the apparent or potential tissue void, cavity, or vessel. For example, axially extending circular contacting element 1b (which is optional) can be formed at the syringe barrel distal closed end. In some embodiments, the axial length of circular contacting element 1b is set to be the same as the difference between the upper and lower limits of the pre-puncture length range of hollow puncture needle 6 (that is, the difference in needle pre-puncture lengths between when hollow puncture needle 6 pierces the syringe barrel distal closed end and when it starts puncturing the organism or tissue). Under this setting, as long as the distal end of hollow puncture needle 6 is still within the axial length range of circular contacting element 1b, early leakage will not happen at needle distal opening 6a. When puncturing, circular contacting element 1b can come into contact with the surface of the organism or tissue first to stabilize the medical puncturing device. Then, pressure can be applied to actuation member 2 to start the puncture operation.

In some embodiments, when the medical puncturing device is in the surface tissue puncture state, that is, when the length of hollow puncture needle 6 extending from the syringe barrel distal closed end is within the surface tissue puncture length range (or when the distal end of hollow puncture needle 6 has pierced the surface tissue but has not yet entered the apparent or potential tissue void, cavity, or vessel), needle body opening 6b is at least partially connected to flowable composition lumen 7. In some embodiments, before the distal end of hollow puncture needle 6 pierces into the apparent or potential tissue void, cavity, or vessel, fluidic communication among flowable composition lumen 7, needle distal opening 6a and needle body opening 6b is established. In some embodiments, the flowable composition in lumen 7 can enter the needle body passageway (via needle body opening 6b) of hollow puncture needle 6 in advance, removing at least part of the air that may be in the needle body passageway, thereby reducing the amount of air entering the apparent or potential tissue void, cavity, or vessel.

In some embodiments, when the distal end of hollow puncture needle 6 starts to pierce into the surface tissue, needle body opening 6b starts to connect with flowable composition lumen 7. In some embodiments, when the distal end of hollow puncture needle 6 pierces into the apparent or potential tissue void, cavity, or vessel, the needle body passageway of hollow puncture needle 6 has already been filled with the flowable composition, thereby eliminating or reducing the possibility of air entering the apparent or potential tissue void, cavity, or vessel.

In some embodiments, when the medical puncturing device is in the fluidic communication state, that is, when the length of hollow puncture needle 6 extending from the syringe barrel distal closed end is within the fluidic communication length range (or when the distal end of hollow puncture needle 6 has pierced into the apparent or potential tissue void, cavity, or vessel), needle body opening 6b has been positioned inside flowable composition lumen 7, achieving maximum flow at needle body opening 6b and thereby increasing injection speed.

The embodiments described herein can be implemented separately or in any suitable combination.

In some embodiments, a device disclosed herein can prevent fluid backflow and/or reverse spill through needle body opening 6b.

In some embodiments, there is a risk for fluid backflow and/or reverse spill from needle body opening 6b when needle distal opening 6a is connected with flowable composition lumen 7, while needle body opening 6b is still at the proximal end of floating seal 3. In some embodiments, there is a risk for fluid backflow and/or reverse spill from needle body opening 6b when needle distal opening 6a is inside the apparent or potential tissue void, cavity, or vessel, while needle body opening 6b is still at the proximal end of floating seal 3. In some embodiments, an elastic sheath 4 covering the outside of hollow puncture needle 6 can be provided within the actuation unit (e.g., elastic movement unit), e.g., between the needle base and floating seal 3. In some embodiments, when needle body opening 6b is at the proximal end of floating seal 3 (e.g., when needle body opening 6b is not connected to flowable composition lumen 7), elastic sheath 4 can keep the needle body opening 6b sealed, thereby effectively avoiding backflow and/or reverse spill of the flowable composition, preventing contamination of the area proximal to floating seal 3, reducing fluid loss, and improving product reliability.

In some embodiments, elastic sheath 4 is not used to seal needle body opening 6b, but simply as an elastic engagement part between floating seal 3 and actuation member 2. In some embodiments, by moving actuation member 2 forward, elastic sheath 4 between floating seal 3 and actuation member 2 can become compressed, thereby forming elastic resilience between floating seal 3 and actuation member 2, which can in turn drive floating seal 3 forward. In some embodiments, the elastic engagement part between floating seal 3 and actuation member 2 can comprise or be a spring 5, which is attached to floating seal 3 and actuation member 2 at its two axial ends, respectively. The attachment at either or both ends of the spring can be direct or indirect. The attachment at either or both ends of the spring can be releasable or not releasable. The spring, the floating seal, and the actuation member (e.g., pressing element) can be separately manufactured and then assembled in any suitable order. Alternatively, any two or more of the spring, the floating seal, and the actuation member (e.g., pressing element) can be integral, e.g., made as one piece. Spring 5 and elastic sheath 4 can be implemented separately or in combination.

In some embodiments, the elastic engagement between floating seal 3 and actuation member 2 can be achieved through other methods besides providing one or more elastic engagement parts. For example, floating seal 3 and actuation member 2 can be provided as a one-piece integrated actuation unit (e.g., elastic movement unit).

In some embodiments, provided herein are devices and methods for implantation into apparent or potential tissue gaps, cavity systems, and vessels using a medical puncturing device disclosed herein. For ease of understanding, a catheter is used as an example for the implanted medical device. In some embodiments, a method disclosed herein comprises using a catheter guiding structure for guiding catheter 11 into the needle body passageway of hollow puncture needle 6. In some embodiments, a catheter guiding structure is provided in a medical puncturing device disclosed herein.

In some embodiments, as shown in FIGS. 6-8, the catheter guiding structure comprises an angled guiding groove 3a, which is provided in or engages floating seal 3 and extends towards hollow puncture needle 6 at an angle. In some embodiments, when flowable composition lumen 7, needle body opening 6b, and needle distal opening 6a are connected, a flowable composition can enter and expand the apparent or potential tissue void, cavity, or vessel. In some embodiments, catheter 11 can be implanted through angled guiding groove 3a, needle body opening 6b, the needle body passageway of hollow puncture needle 6, and needle distal opening 6a into the expanded apparent or potential tissue void, cavity, or vessel.

It should be noted that, angled guiding groove 3a can be provided as a groove through floating seal 3 in a proximal/distal direction, or as a non-through groove formed on a proximal surface of floating seal 3.

In some embodiments, angled guiding groove 3a is a through groove. In some embodiments, the catheter guiding structure further comprises valve 9 provided in or engages angled guiding groove 3a, and the valve may be a one-way valve configured to open and close. In some embodiments, the valve comprises a plurality of leaflets configured to open or close the valve. In some embodiments, in the absence of external force, one-way valve 9 is closed and prevents a flowable composition inside flowable composition lumen 7 from leaking through the valve. In some embodiments, in the presence of an opening force, the plurality of leaflets of the valve can be forced open so that catheter 11 can thread into needle body opening 6b through the opened valve. In some embodiments, the catheter guiding structure further comprises a guiding groove plug configured to be removably inserted in angled guiding groove 3a, and the guiding groove plug can be pulled out when catheter 11 needs to be implanted.

In some embodiments, angled guiding groove 3a is a non-through groove. In some embodiments, the angled guiding groove is punctured directly by catheter 11 to be implanted. In some embodiments, the angled guiding groove is punctured by a piercing component other than the catheter, and catheter 11 can be threaded through the punctured opening into needle body opening 6b.

In some embodiments, to match the guiding direction of angled guiding groove 3a, needle body opening 6b can be provided as an angled opening, which opens obliquely backwards, so that needle body opening 6b can align with angled guiding groove 3a, thereby precisely guiding catheter 11 through the angled guiding groove and into the needle body opening.

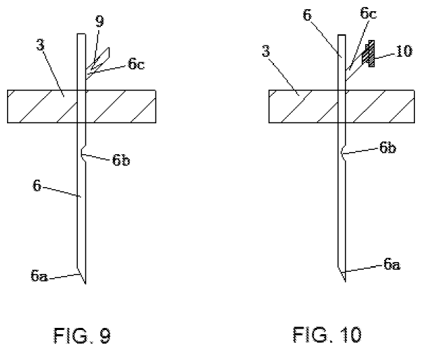

In some embodiments, for example as shown in FIG. 9 and FIG. 10, the catheter guiding structure comprises an angled guiding needle hole 6c which is formed or provided on the body wall of hollow puncture needle 6 and opens obliquely backwards. In some embodiments, angled guiding needle hole 6c remains proximal to floating seal 3, for example, when the medical puncturing device is in a fluidic communication state. In some embodiments, catheter 11 can be threaded into the needle body passageway of hollow puncture needle 6 through angled guiding needle hole 6c. In some embodiments, catheter 11 can be implanted into an apparent or potential tissue void, cavity, or vessel (or an apparent or potential tissue void, cavity, or vessel that has been expanded with a flowable composition) through needle distal opening 6a.

In some embodiments, the catheter guiding structure can further comprise valve 9 provided in or engages angled guiding needle hole 6c, and the valve may be a one-way valve configured to open and close. In some embodiments, the valve comprises a plurality of leaflets configured to open or close the valve. In some embodiments, in the absence of external force, one-way valve 9 is closed and prevents a flowable composition inside flowable composition lumen 7 from leaking through the valve. In some embodiments, in the presence of an opening force, the plurality of leaflets of the valve can be forced open so that catheter 11 can thread into a needle body passageway (which may be connected to or separate from the needle body passageway connecting needle body opening 6b and needle distal opening 6a) through the opened valve and angled guiding needle hole 6c. In some embodiments, the catheter guiding structure can further comprise needle hole plug 10 configured to be removably inserted in angled guiding needle hole 6c, and needle hole plug 10 can be pulled out for the implantation operation of catheter 11 to begin. In some embodiments, guiding needle hole 6c is connected needle distal opening 6a. The needle body passageway connecting needle distal opening 6a and needle body opening 6b can be the same as or separate from the needle body passageway connecting needle distal opening 6a and guiding needle hole 6c. In some embodiments, guiding needle hole 6c is connected to a needle distal opening other than needle distal opening 6a connected to needle body opening 6b. The needle body passageway connecting needle body opening 6b to a needle distal end can be completely separate from the needle body passageway connecting guiding needle hole 6c to a needle distal end. The needle body passageway connecting needle body opening 6b to a needle distal end can be at least partially overlapping or in fluidic communication with the needle body passageway connecting guiding needle hole 6c to a needle distal end.

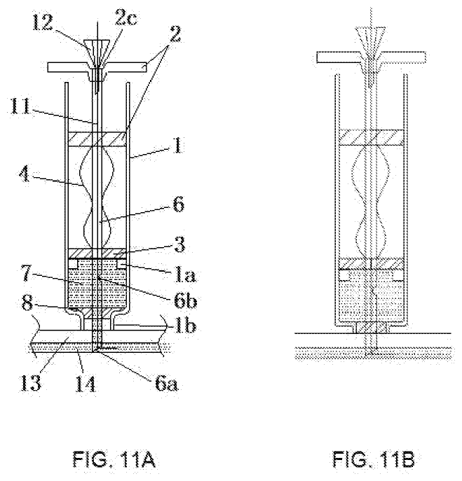

In some embodiments, for example as shown in FIGS. 11A-11B, the catheter guiding structure comprises a central guiding groove 2c that is formed or provided on a proximal surface of actuation member 2. In some embodiments, central guiding groove 2c comprises an aperture or can form an aperture in the center of proximal surface of actuation member 2. In some embodiments, central guiding groove 2c can be punctured to provide an aperture. In some embodiments, a needle proximal opening is provided on hollow puncture needle 6 and is aligned with central guiding groove 2c along the axis. In some embodiments, when catheter 11 needs to be implanted, central guiding groove 2c can be punctured and catheter 11 can be threaded into a needle body passageway (which may be connected to or separate from the needle body passageway connecting needle body opening 6b and needle distal opening 6a) through the punctured opening of central guiding groove 2c and the needle proximal opening of hollow puncture needle 6. In some embodiments, catheter 11 can be implanted into an apparent or potential tissue void, cavity, or vessel (or an apparent or potential tissue void, cavity, or vessel that has been expanded with a flowable composition) through a needle distal opening, such as needle distal opening 6a or a different needle distal opening.

In some embodiments, disclosed herein is a kit comprising components configured to be assembled to form a medical puncturing device disclosed herein.

In some embodiments, the kit for assembling a medical puncturing device comprises a puncture control module and a flowable composition storage module (e.g., a fluid storage module). In some embodiments, the puncture control module and the flowable composition storage module are independently manufactured and/or provided. In some embodiments, the puncture control module comprises a first syringe unit, as well as an actuation unit (e.g., elastic movement unit), and hollow puncture needle 6, which are provided inside a syringe barrel of the first syringe unit. It can be seen based on the embodiments disclosed herein that the puncture control module can further comprise other parts or components, such as elastic sheath 4 and spring 5. In some embodiments, the fluid storage module comprises a second syringe unit, flowable composition lumen 7 which is formed inside a syringe barrel of the second syringe unit, and a module packaging component which is removably provided at the proximal end of the second syringe unit. In some embodiments, a removable connection structure is formed between the first syringe unit and the second syringe unit. In some embodiments, the first syringe unit and the second syringe unit form syringe barrel 1 after being connected with each other. It can be seen based on the embodiments disclosed herein that the fluid storage module can further comprise other parts such as distal seal 8.

In some embodiments, the puncture control module and the fluid storage module can be manufactured, assembled, and/or packaged separately, and then assembled with each other and optionally with other modules, components, and/or parts into the medical puncturing device disclosed herein. In some embodiments, the module packaging component is used to seal the proximal end of flowable composition lumen 7. In some embodiments, when assembling the puncture control module and the fluid storage module, the module packaging component can be removed.

In some embodiments, provided herein is a medical apparatus assembly and a system comprising the same. As shown in FIG. 7 and FIGS. 11A-11B, in some embodiments the medical apparatus assembly comprises catheter 11 and the medical puncturing device comprising the catheter guiding structure disclosed herein. In some embodiments, catheter 11 can be implanted into an apparent or potential tissue void, cavity, or vessel by the medical puncturing device. The medical apparatus assembly described herein can have all of the technical effects provided by the medical puncturing device.

In some embodiments, the medical apparatus assembly comprises hollow auxiliary guiding needle 12, which is matched to be used with the catheter guiding structure. In some embodiments, the needle body passageway diameter of auxiliary guiding needle 12 is large enough to accommodate catheter 11 and allow the catheter to thread in. In some embodiments, during an operation to implant catheter 11, auxiliary guiding needle 12 is connected to the catheter guiding structure so that catheter 11 can sequentially go through the needle body passageway of auxiliary guiding needle 12, the catheter guiding structure, the needle body passageway of hollow puncture needle 6, and then into an apparent or potential tissue void, cavity, or vessel through needle distal opening 6a. In some embodiment, the apparent or potential tissue void, cavity, or vessel is expanded with a flowable composition using a medical puncturing device disclosed herein, prior to the implant of the catheter. In some embodiment, the catheter is implanted as the apparent or potential tissue void, cavity, or vessel is being expanded with a flowable composition using a medical puncturing device disclosed herein. In some embodiment, the catheter is implanted prior to the apparent or potential tissue void, cavity, or vessel being expanded with a flowable composition using a medical puncturing device disclosed herein.

In some embodiments, as shown in FIG. 7, the catheter guiding structure comprises through angled guiding groove 3a and one-way valve 9, which is embedded in angled guiding groove 3a and can be opened and closed. In some embodiments, needle body opening 6b is provided as an angled opening which opens obliquely backwards. In some embodiments, when implanting catheter 11, auxiliary guiding needle 12 is used to open one-way valve 9 so that the auxiliary guiding needle can be positioned inside angled guiding groove 3a. In some embodiments, the distal end of auxiliary guiding needle 12 advances into needle body opening 6b, and catheter 11 can sequentially advance through the needle body passageway of auxiliary guiding needle 12, the needle body passageway of hollow puncture needle 6, and the needle distal opening 6a and then be implanted into an apparent or potential tissue void, cavity, or vessel.

In some embodiments, as shown in FIGS. 11A-11B, the catheter guiding structure comprises a central guiding groove 2c. In some embodiments, a needle proximal opening is formed on hollow puncture needle 6, which is aligned with central guiding groove 2c along its axis. In some embodiments, when implanting catheter 11, central guiding groove 2c can be punctured by auxiliary guiding needle 12, such that auxiliary guiding needle 12 is axially aligned with the proximal opening of hollow puncture needle 6. In some embodiments, catheter 11 is threaded into a needle body passageway of hollow puncture needle 6 by sequentially advancing through a needle body passageway of auxiliary guiding needle 12, and a proximal opening of hollow puncture needle 6, and is then implanted into an apparent or potential tissue void, cavity, or vessel through a needle distal opening such as needle distal opening 6a.

In some embodiments, disclosed herein is a system, comprising: a syringe barrel comprising a proximal end and a distal end; a floating seal in the syringe barrel; a needle base proximal to the floating seal, a piston rod between the floating seal and the needle base, the needle base and the piston rod elastically engaging each other; and a needle in the piston rod, the needle comprising a needle proximal end engaging the needle base and a needle distal end, wherein the needle comprises: (i) a needle distal opening, (ii) a needle body opening between the needle proximal end and the needle distal end, wherein the needle body opening is proximal to the needle distal opening, and (iii) a needle body passageway connecting the needle distal opening and the needle body opening, wherein the needle base is configured to advance the needle distally through the piston rod and toward and/or through the floating seal.

In some embodiments, the floating seal can be fixedly attached to the distal end of the piston rod and form a sliding and sealing engagement with an inner surface of the syringe barrel. In any of the embodiments herein, the needle base can fixedly engage an actuation member (e.g., pressing element), and a spring can engage the actuation member and the piston rod, thereby providing the elastic engagement between the needle base and the piston rod.

In some embodiments, advancement of the needle distally through the piston rod and through the floating seal can occur without moving the floating seal distally, when the needle distal opening is in a tissue or an apparent or potential tissue void, cavity, or vessel providing a higher pressure at the needle distal opening than the pressure at the needle body opening. In some embodiments, the tissue resistance or tissue pressure does not allow injection of the flowable composition through the needle distal opening into the tissue, and the floating seal (as well as the piston rod in embodiments that have one) is not moved distally under a force from the spring, even though the needle can be advanced distally under a force from the pressing shaft. For instance, when the tissue pressure does not allow injection, a needle distal opening of the needle can be in the tissue while a needle body opening is distal to the floating seal and contacting the flowable composition. The floating seal can maintain its position in an axial direction while the needle is further advanced until the needle distal opening reaches an apparent or potential tissue void, cavity, or vessel.

In some embodiments, the floating seal can be moved distally, when the needle distal opening is in a tissue or an apparent or potential tissue void, cavity, or vessel providing a lower pressure at the needle distal opening than the pressure at the needle body opening. In some embodiments, the tissue resistance or tissue pressure allows injection of the flowable composition through the needle distal opening into the tissue, and the floating seal (as well as the piston rod in embodiments that have one) is moved distally under a force from the spring, and the needle does not need to be advanced distally. For instance, when the tissue pressure allows injection, a needle distal opening of the needle can be in the apparent or potential tissue void, cavity, or vessel, while a needle body opening is distal to the floating seal and contacting the flowable composition. The floating seal can be moved distally and the flowable composition is discharged from the needle distal opening while the needle is not further advanced distally.

In some embodiments, provided herein is a method of using a device describe herein for medical penetration. In some embodiments, a preassembled device is provided, as shown in FIG. 17A. In some embodiments, the housing of the preassembled device can be rotated to separate the syringe from the main body of the device. In some embodiments, a proximal portion of the syringe can be in threaded engagement with a distal portion of the housing. For example, the proximal portion of the syringe can comprise threaded grooves on its internal surface which are configured to engage threaded ridges on the outside surface of the distal portion of the housing, as shown in FIG. 17B.

After separation of the syringe, in some embodiments, the proximal end of the piston rod is exposed. A handle can be attached to the piston rod, e.g., via threaded engagement with the proximal end of the piston rod, as shown in FIG. 17C. In some embodiments, an adapter comprising an adapter needle enclosed therein can be attached to the syringe. In some embodiments, the adapter comprises a distal opening and a proximal opening. In some embodiments, the distal end of the syringe (e.g., with the distal seal attached thereto) is inserted into the proximal opening of the adapter, thereby contacting the adapter needle with the distal seal attached to the syringe. In some embodiments, the proximal end of the adapter needle passes through the distal seal attached to the syringe, such that a proximal opening of the adapter needle is inside the internal lumen of the syringe. In some embodiments, a container or a portion thereof containing a flowable composition (e.g., a drug composition) is inserted into the distal opening of the adapter, thereby contacting the adapter needle with the container. In some embodiments, the distal end of the adapter needle inserts into the container, such that a distal opening of the adapter needle is inside the container and capable of establishing a fluid communication between the flowable composition and the internal lumen of the syringe. In some embodiments, the handle is pulled to move the piston rod proximally and draw the flowable composition into the internal lumen of the syringe through the adapter needle, and undesired gas can be expelled by pushing the handle to move the piston rod distally. By pulling and/or pushing the handle, the seal at the distal end of the piston rod and inside the syringe can be placed at a position to set a suitable volume of the flowable composition in the syringe, for example, 0.1 mL or 0.05 mL, as shown in FIG. 17D, and the handle and the adaptor can then be disconnected from the piston rod and the syringe, respectively. The syringe with the flowable composition inside can be connected with the body of the device, e.g., by inserting the syringe needle (e.g., 6 as shown in FIG. 17B) into the piston rod (e.g., 15 as shown in FIG. 17D), inserting the piston rod into the guide tube inside the housing, and screwing the proximal end of the syringe back onto the distal end of the housing, as shown in FIG. 17E. In some embodiments, the control knob can be rotated to advance the pressing shaft in a distal direction, thereby advancing the syringe needle attached to the pressing shaft distally toward and/or through the seal inside the syringe. The syringe needle can be further advanced to pass through the sealing tip, as shown in FIG. 17F, and to pierce into the sclera of the eye. In some embodiments, since the sclera is a dense tissue, the pressure at the distal opening of the syringe needle is greater than the pressure at the body opening of the syringe needle, which can be in fluid communication with the flowable composition inside the syringe; under such conditions, the syringe needle may be further advanced in the sclera without changing the position of the floating seal inside the syringe. In some embodiments, the position of the floating seal inside the syringe is monitored as an operator pushes the pressing shaft to advance the syringe needle. Once the distal opening of the syringe needle is outside the sclera and into the choroid/ciliary body, the pressure at the distal opening of the syringe needle decreases, and the pressure at the body opening of the syringe needle can drive the flowable composition through the needle body passageway and discharge it from the syringe needle distal opening, thereby creating and expanding a suprachoroidal space containing the flowable composition. Since a portion of the flowable composition inside the syringe is discharge, the seal (along with the piston rod) is moved to a more distal position in the syringe. Thus, by observing the movement of the seal, an operator can determine whether the distal opening of the syringe needle has exited a first tissue and reached a second, less dense tissue, e.g., from the sclera into the choroid/ciliary body. In some embodiments, as soon as the seal moves and passes a preset mark or indicator line for volume (e.g., 0.1 mL or 0.05 mL), the advancement of the syringe needle distally is stopped.

In some examples, for instance as shown in FIGS. 17A-17F, the syringe is not prefilled with a flowable material or composition, and the flowable material or composition is drawn from a container into the syringe prior to delivery into a tissue or apparent or potential tissue void, cavity, or vessel.

In some examples, for instance as shown in FIGS. 20A-20B, the syringe of a device disclosed herein can be prefilled with a flowable material or composition. In some embodiments, the syringe (e.g., syringe 1 show in FIG. 16) can be provided in one or more parts. In some embodiments, a container (e.g., a syringe unit), e.g., as shown in FIGS. 20A-20B, can comprise a cylindrical wall sealingly engaging a fixed seal (which can be fixed to the container at a distal end of the container and can be passed through by the needle) and a floating seal (which can move inside the container and can be passed through by the needle), and the space enclosed by the cylindrical wall, the fixed seal and the floating seal can be prefilled with a flowable material or composition. In some embodiments, the device or system can comprise a first syringe unit and the container can be a second syringe unit configured to engage the distal end of the first syringe unit. The container (e.g., syringe unit) can be inserted into or attached to the body (e.g., to the first syringe unit) of the device prior to or after the flowable material or composition is filled into the container (e.g., syringe unit). In some embodiments, the floating seal in the container (e.g., syringe unit) may contact the distal end of the piston rod, thereby establishing an engagement between the piston rod and the floating seal that transmits a force from the spring to the floating seal. The fixed seal at the distal end of the container (e.g., syringe unit) may contact a contacting element at the distal end of the device, and the contacting element can be a distal seal of the syringe as shown in FIG. 20A. In some embodiments, the fixed seal of the container (e.g., syringe unit) also serves as a distal seal of the syringe and/or as a contacting element, e.g., as shown in FIG. 20B. In some embodiments, the container (e.g., syringe unit) can be configured to at least partially insert into a syringe barrel, for instance, as shown in FIGS. 20A-20B. In some embodiments, the fixed seal sealingly engages the container (e.g., syringe unit) which in turn engages an inside wall of the syringe barrel. In some embodiments, the fixed seal sealingly engages both the container (e.g., syringe unit) and an inside wall of the syringe barrel. The engagement between the container (e.g., syringe unit) and the syringe barrel and the engagement between the fixed seal and a wall of the container can comprise any suitable engagement, such as via insertion, a threaded engagement, a non-threaded engagement, engagement secured by a clip, engagement secured by a gland, or any combination thereof.

### IV. Methods and Devices for Drainage from the Eye

Any of the systems and devices disclosed herein can be used for placing a shunt into an eye, comprising: (a) inserting a needle into the eye to form a delivery passageway in the eye, wherein the delivery passageway ends in a target outflow region in the eye; (b) delivering a flowable composition through the needle to form an expanded space in the target outflow region; (c) positioning an inflow end of a shunt (e.g., a micro shunt) in the anterior chamber of the eye and an outflow end of the shunt in the expanded space, wherein the shunt is releasably coupled to the needle; and (d) releasing the needle from the shunt, thereby placing the shunt in the eye to provide fluid communication between the anterior chamber and the target outflow region.

In some embodiments, a shunt disclosed herein can comprise any suitable material. Materials for manufacturing a shunt include but are not limited to medical stainless steel, titanium or titanium alloy, nickel titanium alloy, TPU (thermoplastic polyurethane), e-PTFE (expanded polytetrafluoroethylene), silica gel, hydrogel, PES (polyethersulfone), SIBS (Poly (Styrene-block-IsoButylene-block-Styrene), or any combination thereof. In some embodiments, a material for the shunt has a high biocompatibility, matches the mechanical properties with the eye tissue, and does not damage the eye tissue or cause adverse reaction.

In some embodiments, a shunt disclosed herein can be of any suitable shape. In some embodiments, the shunt is a circular tube. In some embodiments, the shunt comprises a single lumen. In some embodiments, the shunt comprises multiple lumens, e.g., lumens that parallel each other, each extending from one end of the shunt to the other end. In some embodiments, the shape of a cross-section of the shunt is circular, oval, square, or any other suitable shape. In some embodiments, any one or more of the surface(s) of the shunt can be a flat surface or a curved surface.

In some embodiments, a shunt disclosed herein a maker ring and/or a retention ring around the shunt, as shown in FIGS. 18A-18C. In some embodiments, an annular ring around the shunt, such as the retention ring, can be configured to prevent migration of the shunt, e.g., migration of the shunt into the anterior chamber. The shape of the retention ring can include and is not limited to annular, barbed, fin-shaped, or any combination thereof. The structure and dimension of the shunt, including structure and dimension of the marker ring and the retention ring, can be designed to match the ocular tissue structure, thereby effectively draining and reducing intraocular pressure without causing tissue damage and scarring.

In some embodiments, a shunt disclosed herein is about 1.5 mm to about 12 mm in length, for example, about 3 mm, about 4 mm, about 5 mm, or about 6 mm in length. In some embodiments, a shunt disclosed herein is about 0.1 mm to about 1 mm in outer diameter, for example, about 0.25 mm, about 0.3 mm, about 0.35 mm, about 0.4 mm, about 0.45 mm, or about 0.5 mm in outer diameter. In some embodiments, a shunt disclosed herein is about 0.025 mm to about 0.25 mm in inner diameter, for example, about 0.05 mm, about 0.08 mm, about 0.1 mm, about 0.12 mm, or about 0.15 mm in inner diameter. In some embodiments, a marker ring is positioned at about 0.25 mm to about 2.5 mm from one end of the shunt to allow for accurate positioning of the shunt, e.g., for positioning in the anterior chamber. In some embodiments, a shunt disclosed herein is between about 4 mm and about 6 mm in length, between about 0.25 mm and about 0.5 mm in outer diameter, and between about 0.05 mm and about 0.15 mm in inner diameter, whereas the marker ring is positioned at between about 0.5 mm and about 2 mm from the tip of the shunt.

In some embodiments, a shunt disclosed herein can comprise a solid structure, a porous structure, a multi-layer composite structure, a membrane stent structure, or any combination thereof. A solid structure (e.g., a uniform solid structure) is simple and effective and can establish a channel for aqueous drainage, as shown in FIG. 19A. In some embodiments, to avoid or reduce the risk of outlet blockage due to fibrosis and scarring, a micro-porous material can be used to promote bio-integration of surrounding tissues into the material, which can reduce fibrosis and scarring after implant. The porous structure can provide radial muti-channels, increase the drainage efficiency, and reduce the risk of axial outflow channel blockage, as shown in FIG. 19B. In some embodiments, the pore size can be less than 20 microns to prevent excessive growth of tissue or cells into the pore while allowing water to freely pass through the pore. In some embodiments, an inner core or inner layer of a multi-layer composite structure can be designed to provide radial support and establish an effective drainage channel, as shown in FIG. 19C. An outer layer of the multi-layer composite structure can be a porous or fabric layer with pore size less than 20 microns to prevent excessive growth of tissue or cells into the pore, which can lead to channel blockage. In some embodiments, an inner layer of a membrane-covered support (e.g., a membrane stent as shown in FIG. 19D) can comprise a metal stent, and the outer layer can comprise a water permeable membrane. In some embodiments, the metal stent is a hollow support, which can provide sufficient supporting force and flexibility. In some embodiments, the water permeable membrane only allows water molecules to pass through, which can enhance aqueous humor drainage, and prevent tissue or cell growth from blocking the outflow channel.

In some embodiments, provide herein is a method of using a device disclosed herein to deliver a shunt disclosed herein. In some embodiments, the shunt is preloaded in the needle of a delivery system (e.g., a suprachoroidal injection syringe) and implanted into the suprachoroidal space using an ab externo approach described herein. After the tip of the needle of the delivery system punctures the sclera and reaches a choroid/ciliary body layer, a viscoelastic agent is automatically injected to open a suprachoroidal space, and the shunt in the needle is pushed out to the target position, e.g., through a metal wire. The delivery system can be withdrawn to complete the implantation of the shunt. A method disclosed herein can be used for minimally invasive glaucoma surgery (MIGS). Additional details of the device, system, and method are disclosed below.

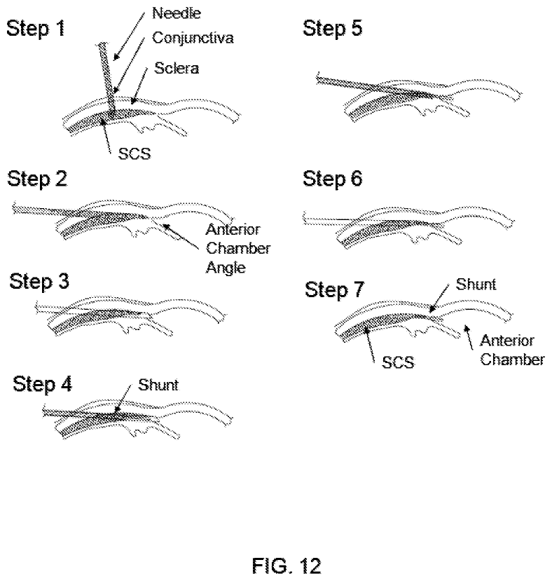

As shown in FIG. 12, Step 1, disclosed herein is a method for placing a shunt into an eye, comprising inserting a needle into the eye through the conjunctiva and the sclera to form a delivery passageway in the eye, wherein the delivery passageway ends between the sclera and the choroid/ciliary body of the eye. A flowable composition such as an viscoelastic material is delivered through the needle to form a suprachoroidal space between the sclera and the choroid/ciliary body. In FIG. 12, Step 2, the needle can be rotated to position a distal end of the needle towards the anterior chamber angle. Because the suprachoroidal space can be expanded by the viscoelastic material, there is more space for the distal end of the needle to be repositioned without damaging the choroid/ciliary body or other surrounding eye tissue. In FIG. 12, Step 3, the needle is moved to pierce the anterior chamber angle with the needle distal end, such that a distal end opening of the needle can be exposed in the anterior chamber. In FIG. 12, Step 4, a shunt can be inserted in the needle (or can be pre-inserted in the needle prior to needle insertion and injection of viscoelastic material) and deployed at the distal end of the needle. In FIG. 12, Step 5, the shunt is positioned through the anterior chamber angle, such that an inflow end of the shunt is in the anterior chamber and an outflow end of the shunt is in the suprachoroidal space. In FIG. 12, Step 6, the needle retreats from the eye, leaving the shunt in the eye to provide fluid communication between the anterior chamber and the suprachoroidal space, as shown in FIG. 12, Step 7.

In some embodiments, a flexible cannula can be used instead of a needle, for example, as shown in FIG. 12, after a flowable material or composition is injected into the suprachoroidal space. In some embodiments, a flowable composition such as an viscoelastic composition can be injected via the syringe needle of an injection device disclosed herein into an eye between the sclera and the choroid/ciliary body, thereby forming a suprachoroidal space containing the flowable composition. In some embodiments, the viscoelastic material forms a bleb or bulge between the sclera and the choroid/ciliary body. In some embodiments, the distal tip of a linear member such as a flexible cannula can be placed in the bleb. In some embodiments, the distal tip of the linear member is advanced through an internal lumen of the syringe needle such that it can placed in the bleb and further advanced between the sclera and the choroid/ciliary body (e.g., towards the anterior chamber angle). In some embodiments, the distal tip of the linear member is inserted through the penetration or injection site of the syringe needle, and advanced towards the bleb along the path created by the syringe needle. The linear member can be inserted through the path created by the syringe needle while the syringe needle remains in the eye. In some embodiments, the linear member parallels the syringe needle, and the two are next to each other in the path created by the syringe needle. In some embodiments, the linear member is inside a lumen of the syringe needle. In some embodiments, the syringe needle is inside a lumen of the linear member.

In some embodiments, once the suprachoroidal space is formed, the syringe needle can be removed from the injection site, leaving the suprachoroidal space filled with the viscoelastic material. The injection site may but does not need to be expanded, for instance, by creating a larger incision from the injection site, and a linear member such as a cannula can be inserted.

In some embodiments, the linear member such as a cannula is a thin, flexible hollow tube with a smooth round tip on the distal end, where the opposite, proximal end can have a hub (e.g., a plastic hub) that can be attached to a syringe. In some embodiments, a shunt can be inserted into the cannula and delivered to a target location through an internal lumen of the cannula. In some embodiments, a shunt can be advanced along the outside surface of the cannula and delivered to a target location. In some embodiments, the cannula comprises a sharp distal tip. In some embodiments, the cannula comprises a blunt distal tip. In some embodiments, the distal end of the cannula opens up a path between structures in tissue, thereby helping dissecting the structures while reducing tissue damage. In some embodiments, the cannula can comprise an opening at its distal end, e.g., for delivering a shunt through the opening.

In some embodiments, the distal tip of the linear member (e.g., a flexible cannula) in the bleb of the flowable composition can be further advanced inside the eye, e.g., between the sclera and the choroid/ciliary body, thereby enlarging the suprachoroidal space towards the front of the eye, e.g., towards the anterior chamber angle. In some embodiments, the linear member is configured to contour to the globe of the eye and the distal tip is configured to pierce into the anterior chamber. In some embodiments, the flowable composition such as an viscoelastic composition provides lubrication of the distal tip of the linear member such that it can slide along the boundary between the sclera and the choroid/ciliary body, reducing the resistance during cannulation and/or reducing the risk of choroidal perforation or the linear member piercing into the vitreous, ciliary body, or other tissues. In some embodiments, the viscoelastic composition forms a protective layer around the distal tip of the linear member, and the protective layer can provide lubrication and guide the direction of cannulation, e.g., towards the anterior chamber angle. In some embodiments, the viscoelastic composition facilitates further dissection of the choroid/ciliary body from the sclera, further expanding the suprachoroidal space towards the front of the eye.

In some embodiments, after the distal end of the linear member reaches a target location, e.g., at a location separated from the anterior chamber angle by a thin tissue, the distal end (or a shunt in or outside the distal end) can pierce the thin tissue, thereby delivering the shunt at the target location such that the shunt connects the anterior chamber and the suprachoroidal space. In some embodiments, once the shunt is delivered, the flexible cannula can be retracted.

In some embodiments, provided herein is a minimally invasive method for placing the shunt into the eye using a needle, without the need to surgically cut open an entire layer of the sclera, or surgically separate the sclera and the choroid/ciliary body, or sewing the cut sclera or conjunctiva after the surgery. Thus, a method disclosed herein can reduce tissue invasion, lower requirements for surgical techniques, and reduce operation time.

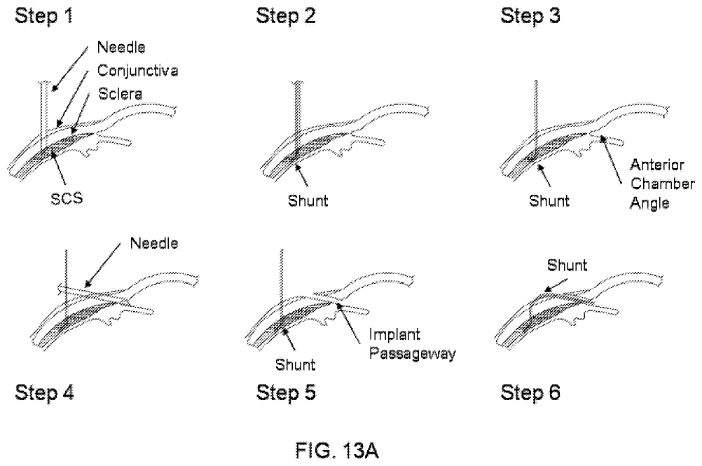

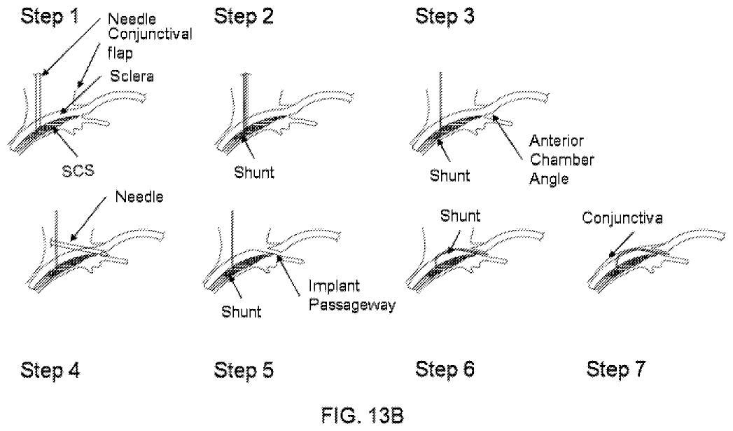

As another example, as shown in FIG. 13A, Step 1, disclosed herein is a method for placing a shunt into an eye, comprising inserting a needle into the eye through the conjunctiva and the sclera to form a delivery passageway in the eye, wherein the delivery passageway ends between the sclera and the choroid/ciliary body of the eye. A flowable composition such as an viscoelastic material is delivered through the needle to form a suprachoroidal space between the sclera and the choroid/ciliary body. In FIG. 13A, Step 2, the needle can be rotated to position a distal end of the needle away from the anterior chamber angle. Because the suprachoroidal space can be expanded by the viscoelastic material, there is more space for the distal end of the needle to be repositioned without damaging the choroid/ciliary body or other surrounding eye tissue. A shunt can be inserted in the needle (or can be pre-inserted in the needle prior to needle insertion and injection of viscoelastic material) and in FIG. 13A, Step 3, the shunt is deployed at the distal end of the needle which is then removed. An outflow end of the shunt is positioned away from the anterior chamber angle, while the other end can be outside the sclera. In FIG. 13A, Step 4, the same needle or a different needle (which can but do not need to be hollow) is used to pierce the sclera, through the suprachoroidal space, and then through the anterior chamber angle of the eye to form an implant passageway, as shown in FIG. 13A, Step 5. In FIG. 13A, Step 6, the other end of the shunt can be inserted into the implant passageway to place an inflow end of the shunt in the anterior chamber, thereby placing the shunt in the eye to provide fluid communication between the anterior chamber and the suprachoroidal space.

In any of the embodiments herein, a portion of the shunt can be outside the sclera. In any of the embodiments herein, the portion of the shunt outside the sclera can be subconjunctival. In any of the embodiments herein, a portion of the shunt can be outside the sclera and the conjunctiva. In any of the embodiments herein, the shunt can comprise a shunt body outflow port that is outside the sclera and subconjunctival and/or a shunt body outflow port that is outside the conjunctiva. In any of the embodiments herein, the shunt can provide fluid communication between the anterior chamber and the suprachoroidal space, and between the anterior chamber and a subconjunctival space. In any of the embodiments herein, the shunt can provide fluid communication between the anterior chamber and the suprachoroidal space, and between the anterior chamber and a space outside the conjunctiva.

For example, as shown in FIG. 13B, Step 1, disclosed herein is a method for placing a shunt into an eye, comprising cutting an opening in the conjunctiva and dissection of the conjunctiva from the sclera to form a conjunctiva flap, followed by inserting a needle into the eye through the sclera to form a delivery passageway in the eye, wherein the delivery passageway ends between the sclera and the choroid/ciliary body of the eye. A flowable composition such as an viscoelastic material is delivered through the needle to form a suprachoroidal space between the sclera and the choroid/ciliary body. In FIG. 13B, Step 2, the needle can be rotated to position a distal end of the needle away from the anterior chamber angle. Because the suprachoroidal space can be expanded by the viscoelastic material, there is more space for the distal end of the needle to be repositioned without damaging the choroid/ciliary body or other surrounding eye tissue. A shunt can be inserted in the needle (or can be pre-inserted in the needle prior to needle insertion and injection of viscoelastic material) and in FIG. 13B, Step 3, the shunt is deployed at the distal end of the needle which is then removed. An outflow end of the shunt is positioned away from the anterior chamber angle, while the other end can be outside the sclera. In FIG. 13B, Step 4, the same needle or a different needle (which can but do not need to be hollow) is used to pierce the sclera, through the suprachoroidal space, and then through the anterior chamber angle of the eye to form an implant passageway, as shown in FIG. 13B, Step 5. In FIG. 13B, Step 6, the other end of the shunt can be inserted into the implant passageway to place an inflow end of the shunt in the anterior chamber, thereby placing the shunt in the eye to provide fluid communication between the anterior chamber and the suprachoroidal space. In FIG. 13B, Step 7, the opening of the conjunctival flap is sewn to cover the portion of the shunt outside the sclera. In any of the embodiments herein, the portion of the shunt outside the sclera can comprise a shunt body outflow port, and the shunt body outflow port can be subconjunctival (e.g., covered by the sewn conjunctiva flap).

In any of the embodiments herein, the inflow end, the outflow end, and/or the shunt body outflow port can comprise one or more valves, such as one-way valves, for example, to control flow of fluid from the anterior chamber. For instance, fluid flow can be controlled such that fluid flows from the anterior chamber to the outflow end (e.g., in the suprachoroidal space or the subconjunctival space) and/or the shunt body outflow port (e.g., in the subconjunctival space or outside the conjunctiva), but not from the outflow end or the shunt body outflow port to the inflow port in the anterior chamber.

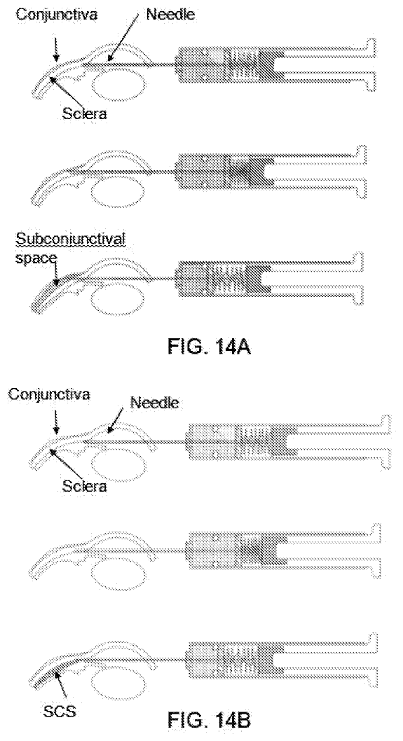

In some examples, as shown in FIG. 14A, a medical puncture device disclosed herein can be used is an ab interno method for placing a shunt into an eye. The needle of the medical puncture device can be inserted into the eye, through the cornea, across the anterior chamber, and to a subconjunctival space. A flowable composition such as an viscoelastic material is delivered through the needle into the subconjunctival space. The subconjunctival space can be expanded by the viscoelastic material, to avoid or reduce risk of the needle piercing the conjunctiva during the injection process. Then a shunt can be deployed through the hollow needle, in order to position an outflow end of the shunt in the subconjunctival space, and after the needle is removed, an inflow end of the shunt is placed in the anterior chamber, in order to provide fluid communication between the anterior chamber and the subconjunctival space. Compared to certain other ab interno methods, the presently disclosed method enables control of the injection and expansion of the subconjunctival space to reduce risk of the needle piercing the conjunctiva.

In some examples, as shown in FIG. 14B, a medical puncture device disclosed herein can be used is an ab interno method for placing a shunt into an eye, by inserting a needle into the eye, through the cornea, across the anterior chamber, and to a suprachoroidal space (SCS). A flowable composition such as an viscoelastic material is delivered through the needle into the SCS. The SCS can be expanded by the viscoelastic material. Then a shunt can be deployed through the hollow needle, in order to position an outflow end of the shunt in the SCS, and after the needle is removed, an inflow end of the shunt is placed in the anterior chamber, in order to provide fluid communication between the anterior chamber and the SCS. In some embodiments, the needle has a blunt piercing end. Compared to certain other ab interno methods, the presently disclosed method enables control of the injection and expansion of the SCS to reduce risk of the needle piercing the sclera and/or the conjunctiva. In some embodiments, when the distal opening of the needle is placed in the anterior chamber angle and/or ciliary body, the viscoelastic material is not injected; and when the distal opening of the needle is placed between the sclera and the choroid/ciliary body, the viscoelastic material is injected into the potential cavity, thereby expanding the suprachoroidal space.

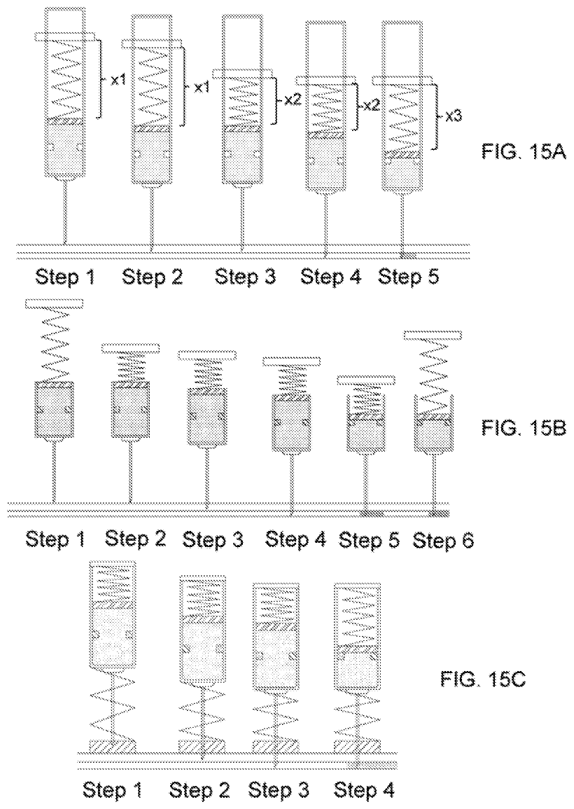

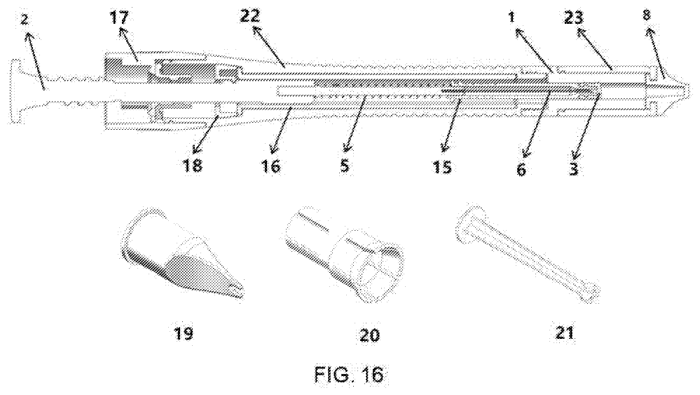

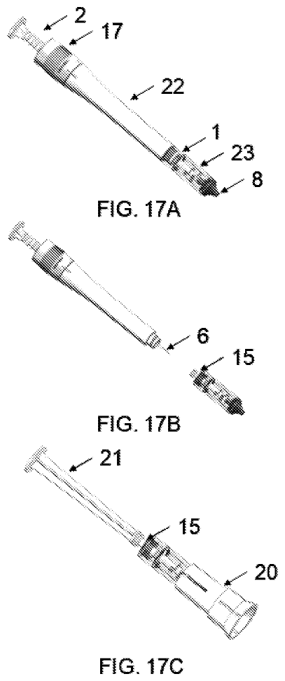

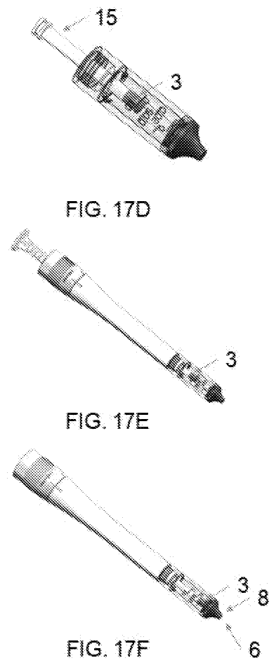

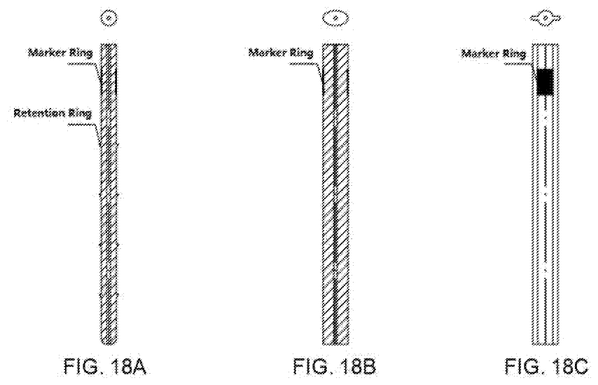

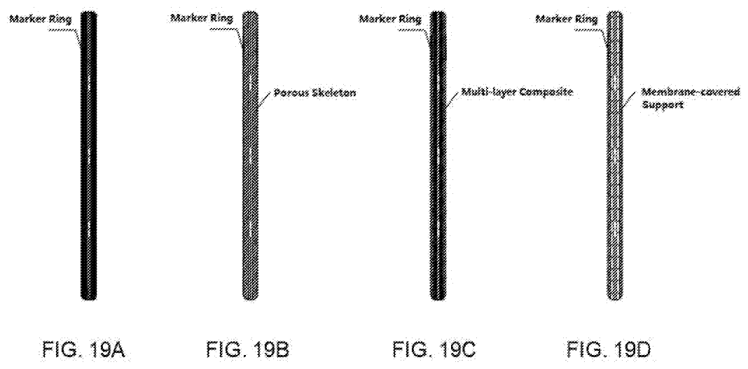

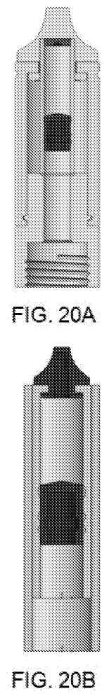

It should be appreciated that any suitable medical puncture device, including but not limited to those described herein in connection with the figures, may be used in a method for drainage from an eye disclosed herein. For instance, a medical puncture device shown in FIG. 15A may be used. In some embodiments, the medical puncture device comprises a syringe barrel comprising a proximal end and a distal end; a floating seal in the syringe barrel; a puncture member such as a needle at the distal end of the syringe barrel, wherein the puncture member is not attached to the floating seal; and an actuation member configured to elastically engage the floating seal via an energy storage member such as a spring or the like and/or another suitable elastic member. In some embodiments, the puncture member comprises a distal end opening configured to form a fluidic communication with a lumen in the syringe barrel containing a flowable composition. In some embodiments, the medical puncture device further comprises a stopper in the syringe barrel, between the floating seal and the distal end of the syringe barrel. As shown in FIG. 15A, Step 1, the medical puncture device is in an initial state where the distal end opening of the puncture member has not entered a tissue of a subject, and the distance between the actuation member and the floating seal is x₁. In FIG. 15A, Step 2, the distal end opening of the puncture member has entered a relatively dense tissue (e.g., the sclera, anterior chamber angle, or ciliary body), where the distance between the actuation member and the floating seal remains the same (x₁). In FIG. 15A, Step 3, the distal end opening of the puncture member remains in the relatively dense tissue, when the energy storage member is compressed, e.g., by reducing the distance between the actuation member and the floating seal from x₁ to x₂. This way, the energy storage member applies a force on the floating seal and maintains the force. Through the flowable composition and the distal opening of the puncture member, a pressure is in turn applied to the relatively dense tissue. Due to the tissue density, the relatively dense tissue applies a back pressure on the distal opening of the puncture member, thereby preventing discharge of the flowable composition into the tissue. In FIG. 15A, Step 4, the puncture member is advanced distally into a less dense tissue, such as an apparent or potential tissue void, cavity, or vessel (for instance, the SCS or the subconjunctival space). In some embodiments, due to the decrease in tissue density, the back pressure on the distal opening of the puncture member becomes less than the pressure of the flowable composition, thereby allowing release of the flowable composition into the less dense tissue, such as the apparent or potential tissue void, cavity, or vessel. As the flowable composition is discharged from the distal end opening of the puncture member, energy in the energy storage member is released, thereby increasing the distance between the actuation member and the floating seal from x₂ to x₃, as shown in FIG. 15A, Step 5. Distal movement of the floating seal in the syringe barrel may be stopped by the stopper, for example, in order to control the volume of the flowable composition delivered into the less dense tissue.

Another example is shown in FIG. 15B, Step 1, where the medical puncture device is in an initial state where the distal end opening of the puncture member has not entered a tissue of a subject, and in FIG. 15B, Step 2, the energy storage member can be compressed, whereas the distal end opening of the puncture member remains outside a tissue and the floating seal is not advanced distally to discharge the flowable composition from the distal end opening. In FIG. 15B, Step 3, the distal end opening of the puncture member has entered a relatively dense tissue (e.g., the sclera, anterior chamber angle, or ciliary body). The energy storage member applies a force on the floating seal and maintains the force. Through the flowable composition and the distal opening of the puncture member, a pressure is in turn applied to the relatively dense tissue. Due to the tissue density, the relatively dense tissue applies a back pressure on the distal opening of the puncture member, thereby preventing discharge of the flowable composition into the tissue. In FIG. 15B, Step 4, the distal end opening of the puncture member starts to enter a less dense tissue, such as an apparent or potential tissue void, cavity, or vessel (for instance, the SCS or the subconjunctival space), whereas the energy storage member remains compressed. In FIG. 15B, Step 5, due to the decrease in tissue density, the back pressure on the distal opening of the puncture member becomes less than the pressure of the flowable composition, thereby allowing release of the flowable composition into the less dense tissue. Energy in the energy storage member is released, as the flowable composition is discharged from the distal end opening of the puncture member. In some embodiments, distal movement of the floating seal in the syringe barrel may be stopped by the stopper to stop the flow of the flowable composition. This way, the volume of the flowable composition delivered into the less dense tissue may be controlled. The force applied onto the actuation member may be released as shown in FIG. 15B, Step 6.

Yet another example is shown in FIG. 15C. In some embodiments, the medical puncture device comprises a syringe barrel comprising a proximal end and a distal end; a floating seal in the syringe barrel; a puncture member such as a needle at the distal end of the syringe barrel, wherein the puncture member is not attached to the floating seal; and an energy storage member configured to elastically engage the floating seal and the proximal end of the syringe barrel. In some embodiments, the medical puncture device further comprises a stopper in the syringe barrel, between the floating seal and the distal end of the syringe barrel. In some embodiments, the medical puncture device comprises a contact member. In FIG. 15C, Step 1, the medical puncture device is in an initial state where the distal end opening of the puncture member in the contact member which prevents discharge of the flowable composition from the distal end opening. The energy storage member applies a force onto the floating seal, and through the flowable composition and the distal opening of the puncture member, a pressure is in turn applied to the contact member. Due to the density of the contact member, the back pressure on the distal opening of the puncture member prevents leakage of the flowable composition from the syringe barrel. In FIG. 15C, Step 2, the distal end opening of the puncture member has entered a relatively dense tissue (e.g., the sclera, anterior chamber angle, or ciliary body), and the back pressure of the relatively dense tissue on the distal opening prevents leakage of the flowable composition into the tissue. In FIG. 15C, Step 3, the distal end opening of the puncture member starts to enter a less dense tissue, such as an apparent or potential tissue void, cavity, or vessel (for instance, the SCS or the subconjunctival space). In FIG. 15C, Step 4, due to the decrease in tissue density, the back pressure on the distal opening of the puncture member becomes less than the pressure of the flowable composition, thereby allowing release of the flowable composition into the less dense tissue. Energy in the energy storage member is released, as the flowable composition is discharged from the distal end opening of the puncture member. In some embodiments, distal movement of the floating seal in the syringe barrel may be stopped by the stopper to stop the flow of the flowable composition. This way, the volume of the flowable composition delivered into the less dense tissue may be controlled.

The exemplary embodiments and optional implementations of the present disclosure are described in detail above in combination with the figures. However, the present disclosure is not limited to the details described in the embodiments described above. Simple variants can be applied to the embodiments of the present disclosure, all of which are within the scope of the present disclosure.

It should be noted that, each of the technical features described in the embodiments above, when not in conflict, can be combined in any reasonable manner. To avoid unnecessary repetition, the possible combinations are not described separately in the embodiments.

Additionally, the different implementations of the embodiments of the present disclosure can be freely combined. As long as they do not go against the ideas of the present disclosure, they should also be considered part of this disclosure.

1. A method for placing a shunt into an eye, comprising:
(a) inserting a needle into the eye to form a delivery passageway in the eye, wherein the delivery passageway ends in a target outflow region in the eye;
(b) delivering a flowable composition through the needle to form an expanded space in the target outflow region;
(c) positioning an inflow end of a shunt in the anterior chamber of the eye and an outflow end of the shunt in the expanded space, wherein the shunt is releasably coupled to the needle; and
(d) releasing the needle from the shunt,
thereby placing the shunt in the eye to provide fluid communication between the anterior chamber and the target outflow region.

2. The method of claim 1, wherein the needle pierces the sclera.

3. The method of claim 1 or 2, wherein the method comprises cutting open a region in the conjunctiva, optionally prior to the needle piercing the sclera.

4. The method of claim 1 or 2, wherein the needle pierces the conjunctiva and the sclera, and wherein the method does not comprise cutting open a region in the conjunctiva.

5. The method of any of claims 1-4, wherein the target outflow region is between the sclera and the choroid/ciliary body, and the expanded space is a suprachoroidal space.

6. The method of claim 5, wherein the positioning step comprises positioning a distal end of the needle in the suprachoroidal space and towards the anterior chamber angle.

7. The method of any of claims 1-6, wherein:
the shunt is within the needle, optionally wherein the shunt is in a needle body passageway of the needle, or
the shunt forms a sleeve around the needle.

8. The method of claim 7, wherein the positioning step comprises advancing the shunt in/around the needle to a distal end of the needle.

9. The method of claim 8, wherein the advancing comprises pushing the shunt in/around the needle using a guidewire.

10. The method of any of claims 7-9, wherein the positioning step comprises piercing the anterior chamber angle with a distal end of the needle and/or the shunt.

11. The method of any of claims 7-10, wherein the releasing step comprises removing the needle and/or the guidewire from the eye, leaving the inflow end of the shunt in the anterior chamber and the outflow end of the shunt in the suprachoroidal space.

12. The method of any of claims 7-11, wherein the shunt is coupled to the needle prior to or after the inserting step.

13. The method of any of claims 7-12, wherein the shunt is coupled to the needle prior to or after delivering the flowable composition.

14. The method of any of claims 1-6, wherein the shunt is releasably coupled to a distal end of the needle.

15. The method of claim 14, wherein the positioning step comprises positioning the shunt towards the anterior chamber angle.

16. The method of claim 14 or 15, wherein the positioning step comprises advancing the needle to pierce the anterior chamber angle with a distal end of the shunt.

17. The method of any of claims 14-16, wherein the releasing step comprises removing the needle, leaving the inflow end of the shunt in the anterior chamber and the outflow end of the shunt in the suprachoroidal space.

18. The method of claim 5, wherein the positioning step comprises positioning a distal end of the needle in the suprachoroidal space and away from the anterior chamber angle.

19. The method of claim 18, wherein:
the shunt is within the needle, optionally wherein the shunt is in a needle body passageway of the needle, or
the shunt forms a sleeve around the needle.

20. The method of claim 19, wherein the positioning step comprises advancing the shunt in/around the needle to the distal end of the needle.

21. The method of claim 20, wherein the advancing comprises pushing the shunt in/around the needle using a guidewire.

22. The method of any of claims 19-21, wherein the positioning step comprises positioning the outflow end of the shunt in the suprachoroidal space and away from the anterior chamber angle.

23. The method of any of claims 19-22, wherein the positioning step comprises removing the needle from the eye, leaving the outflow end of the shunt in the suprachoroidal space.

24. The method of any of claims 19-23, further comprising piercing the anterior chamber angle to form an implant passageway.

25. The method of claim 24, wherein the inflow end of the shunt is positioned through the implant passageway in the anterior chamber.

26. The method of claim 24 or 25, wherein the implant passageway is formed using the same needle or a different piercing element.

27. The method of claim 26, wherein the same needle or different piercing element pierces through the conjunctiva, the sclera, the suprachoroidal space, and the anterior chamber angle.

28. The method of claim 26 or 27, wherein the needle is inserted into the eye at a first entry point, and the same needle or different piercing element is inserted into the eye at a second entry point different from the first entry point to form the implant passageway.

29. The method of claim 28, wherein the shunt comprises a portion between the first and second entry points that is outside the sclera.

30. The method of claim 28 or 29, wherein the shunt comprises a portion between the first and second entry points that is outside the sclera and the conjunctiva.

31. The method of claim 29, wherein the portion outside the sclera is subconjunctival.

32. The method of claim 31, wherein the method comprises cutting an opening in the conjunctiva, dissecting the conjunctiva from the sclera to form a conjunctiva flap, and after the inflow end of the shunt is positioned through the implant passageway in the anterior chamber, sewing the opening of the conjunctival flap to cover the portion of the shunt outside the sclera.

33. The method of any of claims 29-32, wherein the portion of the shunt outside the sclera comprises a shunt body outflow port, optionally wherein the shunt body outflow port is subconjunctival.

34. The method of any of claims 29-33, further comprising applying an antimetabolite between the conjunctival flap and the sclera to modulate postoperative scarring.

35. The method of any of claims 1-34, wherein the needle is inserted into the eye ab externo.

36. The method of any of claims 1-34, wherein the needle is inserted into the eye ab interno.

37. The method of any of claims 1-4, wherein the target outflow region is between the conjunctiva and the sclera, and the expanded space is a subconjunctival space, optionally wherein the subconjunctival space is a subconjunctival bleb.

38. The method of claim 37, wherein:
the shunt is within the needle, optionally wherein the shunt is in a needle body passageway of the needle;
the shunt forms a sleeve around the needle; or
the shunt is releasably coupled to a distal end of the needle.

39. A method for placing a shunt into an eye, comprising:
(a) inserting a needle into the eye to form a delivery passageway in the eye, wherein the delivery passageway ends between the sclera and the choroid/ciliary body of the eye;
(b) delivering a flowable composition through the needle to form a suprachoroidal space;
(c) piercing the anterior chamber angle of the eye with a distal end of the needle and/or a shunt releasably coupled thereto;
(d) positioning the shunt through the anterior chamber angle such that an inflow end of the shunt is in the anterior chamber and an outflow end of the shunt is in the suprachoroidal space; and
(e) removing the needle from the eye,
thereby placing the shunt in the eye to provide fluid communication between the anterior chamber and the suprachoroidal space.

40. A method for placing a shunt into an eye, comprising:
(a) inserting a needle into the eye to form a delivery passageway in the eye, wherein the delivery passageway ends between the sclera and the choroid/ciliary body of the eye;
(b) delivering a flowable composition through the needle to form a suprachoroidal space;
(c) using the needle to position an outflow end of a shunt in the suprachoroidal space and away from the anterior chamber angle;
(d) piercing the anterior chamber angle of the eye to form an implant passageway; and
(e) positioning an inflow end of the shunt in the anterior chamber through the implant passageway,
thereby placing the shunt in the eye to provide fluid communication between the anterior chamber and the suprachoroidal space.

41. The method of claim 40, wherein a portion of the shunt is outside the sclera.

42. The method of claim 41, wherein the portion of the shunt outside the sclera is subconjunctival.

43. The method of any of claims 40-42, wherein a portion of the shunt is outside the sclera and the conjunctiva.

44. The method of any of claims 40-43, wherein the shunt comprises a shunt body outflow port that is outside the sclera and subconjunctival and/or a shunt body outflow port that is outside the conjunctiva.

45. The method of claim 44, wherein the shunt provides fluid communication between the anterior chamber and the suprachoroidal space, and between the anterior chamber and a subconjunctival space.

46. The method of claim 44 or 45, wherein the shunt provides fluid communication between the anterior chamber and the suprachoroidal space, and between the anterior chamber and a space outside the conjunctiva.

47. An ab interno method for placing a shunt into an eye, comprising:
(a) inserting a needle and/or a shunt releasably coupled thereto through the cornea, across the anterior chamber, and to a suprachoroidal space or a subconjunctival space;
(b) delivering a flowable composition through the needle and/or the shunt into the suprachoroidal space or the subconjunctival space;
(c) positioning an inflow end of the shunt in the anterior chamber and an outflow end of the shunt in the suprachoroidal space or the subconjunctival space; and
(d) removing the needle from the eye,
thereby placing the shunt in the eye to provide fluid communication between the anterior chamber and the suprachoroidal space or the subconjunctival space.

48. The method of any of claims 1-47, wherein the shunt comprises a pharmaceutical or biological agent.

49. The method of any of claims 1-48, comprises using a device comprising:
a syringe barrel comprising a proximal end and a distal end;
a floating seal in the syringe barrel;
a needle base proximal to the floating seal, wherein the floating seal and the needle base elastically engage each other; and
the needle, wherein the needle comprises:
(i) a needle proximal end engaging the needle base;
(ii) a needle distal end;
(iii) a needle distal opening;
(iv) a needle body opening between the needle proximal end and the needle distal end, wherein the needle body opening is proximal to the needle distal opening; and
(v) a needle body passageway connecting the needle distal opening and the needle body opening,
wherein the needle base is configured to advance the needle distally toward and/or through the floating seal.

50. The method of claim 49, wherein the floating seal separates a proximal lumen and a distal lumen in the syringe barrel, and wherein the distal lumen comprises the flowable composition.

51. The method of claim 50, wherein the needle base is configured to advance the needle distally such that the needle distal opening is in the sclera, whereas the needle body opening is in the distal lumen comprising the flowable composition.

52. The method of claim 51, wherein the sclera is capable of preventing discharge of the flowable composition in the distal lumen through the needle distal opening, optionally wherein the back pressure at the needle distal opening in the sclera is no less than the pressure in the distal lumen.

53. The method of claim 52, wherein the needle base is configured to advance the needle distally such that the needle body opening is in the distal lumen while the needle distal opening is between the sclera and an adjacent tissue.

54. The method of claim 53, wherein the needle distal opening is between the sclera and the choroid/ciliary body, and the flowable composition is delivered through the needle to the suprachoroidal space.

55. The method of claim 53, wherein the needle distal opening is between the sclera and the conjunctiva, and the flowable composition is delivered through the needle to the subconjunctival space.

56. The method of any of claims 1-55, wherein the flowable composition comprises a liquid, a solution, a suspension, a gel, an oil, an ointment, an emulsion, a cream, a foam, a lotion, and/or a paste.

57. The method of any of claims 1-56, wherein the shunt is configured to advance distally through or along the needle and be exposed at a distal end of the needle when the needle reaches the target outflow region.

58. A system comprising the needle, the shunt, and the flowable composition for use in the method of any of claims 1-57.

59. A system for placing a shunt into an eye, comprising:
a syringe barrel comprising a proximal end and a distal end;
a floating seal in the syringe barrel;
a needle base proximal to the floating seal, wherein the floating seal and the needle base elastically engage each other;
a needle for insertion into the eye, the needle comprising:
(i) a needle proximal end engaging the needle base;
(ii) a needle distal end;
(iii) a needle distal opening;
(iv) a needle body opening between the needle proximal end and the needle distal end, wherein the needle body opening is proximal to the needle distal opening; and
(v) a needle body passageway connecting the needle distal opening and the needle body opening,
wherein the needle base is configured to advance the needle distally toward and/or through the floating seal; and
the shunt configured to releasably couple to the needle.

60. The system of claim 58 or 59, wherein the shunt is an intraocular shunt within the needle.

61. The method or system of any of claims 1-60, wherein the shunt comprises a solid structure, a porous structure, a multi-layer composite structure, a membrane stent structure, or any combination thereof.

62. The method or system of any of claims 1-61, wherein the shunt comprises one or more annular rings on a sidewall of the shunt.

63. The method or system of claim 63, wherein the shunt comprises a marker ring and a retention ring on the sidewall of the shunt.

64. The method or system of any of claims 1-63, wherein the shunt comprises multiple channels each extending from one end of the shunt to the other end.
